(19) United States
(12) Patent Application Publication
(10) Pub. No.: US 2012/0203245 A1
(43) Pub. Date: Aug. 9, 2012

(54) NERVE STIMULATOR

(75) Inventors: Hiroyuki IMABAYASHI, Tokyo (JP); Kazuo SHIMIZU, Tokyo (JP); Hinako KAJI, Tokyo (JP); Masamichi NOGUCHI, Tokyo (JP)

(73) Assignee: OLYMPUS CORPORATION, Tokyo (JP)

(21) Appl. No.: 13/364,642

(22) Filed: Feb. 2, 2012

(30) Foreign Application Priority Data
Feb. 7, 2011 (JP) P2011-024269
Aug. 25, 2011 (JP) P2011-183421
Nov. 2, 2011 (JP) P2011-241373

(51) Int. Cl. A61B 17/00; A61N 1/05

(52) U.S. Cl. 606/129; 607/119

(57) ABSTRACT

A nerve stimulation electrode implantation system includes: an introduction portion that has a cylindrical main body which has an internal cavity, and an incision component which has a blunt dissection portion that bluntly dissects biological tissue, and which is formed so as to be transparent, and which is attached to a distal end portion of the main body; an observation portion that is inserted into the introduction portion such that it is able to observe the periphery of the incision component through the incision component; and a peeling portion that is positioned such that it is able to rotate around its own axis and is able to move relatively in this axial direction relative to the introduction portion, and that removes peripheral tissue from the periphery of the nerve tissue.

### BACKGROUND OF THE INVENTION

1. Field of the Invention

The present invention relates to a nerve stimulation electrode implantation system that is used to implant a nerve-stimulating electrode within a body and a defibrillation electrode implantation system that is used to implant a cardiac defibrillation electrode within a body.

Priority is claimed on Japanese Patent Application Nos. 2011-024269, filed Feb. 7, 2011, 2011-241373, filed Nov. 2, 2011, and 2011-183421, filed Aug. 25, 2011, the contents of which are incorporated herein by reference.

2. Description of Related Art

Conventionally, implantable pulse generators for performing treatment are known that impart electrical stimulation either directly or indirectly to biological tissue (i.e., linear tissue) such as nerve tissue and muscle and the like such as nerve stimulators, pain alleviation devices, epilepsy treatment devices, and muscle stimulators. These implantable pulse generators have an internal power supply and, normally, are used by being embedded within a body together with a stimulation electrode that transmits electrical stimulation.

Generally, stimulation electrodes are provided with: at least one electrode that is used to impart electrical stimulation to biological tissue, or that is used to detect electrical excitation generated in biological tissue; an electrical connector that is used to provide a connection with an implantable pulse generator; and a lead portion that is provided between the electrode and the implantable pulse generator and is used to transmit electrical stimulation.

For example, in Japanese Unexamined Patent Application, First Publication No. 2004-173790, an embedded type of cardiac treatment device is disclosed that, if a patient's heart is afflicted with bradycardia, stimulates the heart so as to raise the heart rate, or that, if a patient's heart is afflicted with tachycardia or arrhythmia, stimulates the vagus nerve so as to lower the heart rate. In Japanese Unexamined Patent Application, First Publication No. 2004-173790, the cardiac stimulation electrode is placed within the heart muscle or in the atrium. The neck region or a position in the center of the right external carotid artery are preferred for the location where the nerve stimulation electrode is to be wound and left implanted.

In addition, for example, in Published Japanese Translation No. 2005-523786 of the PCT International Publication, a defibrillation system is disclosed that is provided with an implantable automated defibrillator as an implantable pulse generator. This defibrillation system has a pair of subcutaneous patch electrodes (defibrillation electrodes) that are suitable for subcutaneous embedding, and a pair of electrical leads that connect together the respective subcutaneous patch electrodes and the defibrillator.

One of the pair of subcutaneous patch electrodes is implanted in a subcutaneous pocket which is formed in the anterior part of the chest of the patient on the outer side of the thorax. The other subcutaneous patch electrode is implanted in a subcutaneous pocket which is formed on the dorsal side of the patient on the outer side of the thorax. Consequently, there is no need to perform a thoracotomy in order to implant the electrodes and they can be attached with less-invasive to the patient.

### SUMMARY OF THE INVENTION

A nerve stimulation electrode implantation system according to a first aspect of the present invention is a nerve stimulation electrode implantation system that is used to implant a nerve stimulation electrode in intended nerve tissue, and that includes: an introduction portion that has a cylindrical main body which has an internal cavity, and an incision component which has a blunt dissection portion that bluntly dissects biological tissue, and which is formed so as to be transparent, and which is attached to a distal end portion of the main body; an observation portion that is inserted into the introduction portion such that it is able to observe the periphery of the incision component through the incision component; and a peeling portion that is positioned such that it is able to rotate around its own axis and is able to move relatively in this axial direction relative to the introduction portion, and that removes surrounding tissue from the periphery of the nerve tissue.

The nerve stimulation electrode implantation system according to a second aspect of the present invention is further provided with an electrode operating component that is inserted into the nerve stimulation electrode and moves the nerve stimulation electrode forwards and backwards and also in rotation, and, when the electrode operating component is inserted therein, for the nerve stimulation electrode to be introduced along the introduction portion as far as the nerve tissue.

In the nerve stimulation electrode implantation system according to a third aspect of the present invention, the nerve stimulation electrode implantation system according to either of the first or second aspects for the main body includes: a first internal cavity inside which the observation portion is inserted such that it can move forwards and backwards; and a second internal cavity inside which the peeling portion is inserted such that it can move forwards and backwards.

In the nerve stimulation electrode implantation system according to a fourth aspect of the present invention, the main body includes an electrode cavity inside which the nerve stimulation electrode which has the electrode operating component inserted inside it is inserted such that it can move forwards and backwards.

In the nerve stimulation electrode implantation system according to a fifth aspect of the present invention, the nerve stimulation electrode implantation system according to any of the first through fourth aspects for the field of view of the observation portion faces towards the distal end side of the introduction portion.

The nerve stimulation electrode implantation system according to a sixth aspect of the present invention is further provided with a determination electrode that is provided on at least one of an external surface of the distal end portion of the main body and an external surface of the incision portion.

In the nerve stimulation electrode implantation system according to a seventh aspect of the present invention, in the nerve stimulation electrode implantation system according to the sixth aspect, the determination electrode is formed around the entire circumference of the main body.

In the nerve stimulation electrode implantation system according to an eighth aspect of the present invention, in the nerve stimulation electrode implantation system according to the sixth aspect, the determination electrode is formed in only a portion in the circumferential direction of the main body.

In the nerve stimulation electrode implantation system according to a ninth aspect of the present invention, in the nerve stimulation electrode implantation system according to the eighth aspect, an index mark is formed at the same position in the circumferential direction of the internal cavity as the determination electrode on at least one of the proximal end portion of the main body and the incision component.

In the nerve stimulation electrode implantation system according to a tenth aspect of the present invention, in the nerve stimulation electrode implantation system according to the sixth aspect, the determination electrode is formed so as to extend in a longitudinal direction of the main body.

A nerve stimulation electrode implantation system according to an eleventh aspect of the present invention includes: an introduction portion that has a cylindrical main body which has an internal cavity, an incision component having a blunt dissection portion that bluntly dissects biological tissue, and which is attached to a distal end portion of the main body and is formed so as to be transparent, and a determination electrode which is provided on at least one of an external surface of the distal end portion of the main body and an external surface of the incision portion; an observation portion that is inserted into the introduction portion such that it is able to observe the periphery of the incision component through the incision component; and a peeling portion that is positioned such that it is able to rotate around its own axis and is able to move relatively in this axial direction relative to the introduction portion, and that removes surrounding tissue from the periphery of the nerve tissue.

In the nerve stimulation electrode implantation system according to a twelfth aspect of the present invention, the nerve stimulation electrode implantation system according to the eleventh aspect, the determination electrode is formed around the entire circumference of the main body.

In the nerve stimulation electrode implantation system according to a thirteenth aspect of the present invention, in the nerve stimulation electrode implantation system according to the eleventh aspect, the determination electrode is formed in only a portion in the circumferential direction of the main body.

In the nerve stimulation electrode implantation system according to a fourteenth aspect of the present invention, in the nerve stimulation electrode implantation system according to the thirteenth aspect, an index mark is formed at the same position in the circumferential direction of the internal cavity as the determination electrode on at least one of the proximal end portion of the main body and the incision component.

In the nerve stimulation electrode implantation system according to a fifteenth aspect of the present invention, in the nerve stimulation electrode implantation system according to the eleventh aspect, the determination electrode is formed so as to extend in a longitudinal direction of the main body.

In the nerve stimulation electrode implantation system according to a sixteenth aspect of the present invention, in the nerve stimulation electrode implantation system according to the eleventh aspect, there is provided a monitoring portion that measures the state of a living organism and displays the measurement results.

In the nerve stimulation electrode implantation system according to a seventeenth aspect of the present invention, the nerve stimulation electrode implantation system according to the sixteenth aspect for the measurement results is an electrocardiogram.

In the nerve stimulation electrode implantation system according to an eighteenth aspect of the present invention, the nerve stimulation electrode implantation system according to the sixteenth aspect for the measurement results is an electromyogram.

A defibrillation electrode according to a nineteenth aspect of the present invention includes: an electrode that has an electrode surface; a lead portion whose distal end side is connected to the electrode portion, and that is able to transmit to the electrode portion rotation movement which is applied to itself; an index mark that is formed on a proximal end side of the lead portion on a portion in the circumferential direction of the lead portion; and a connector that is provided on a proximal end side of the lead portion, and that is connected to an implantable defibrillator, wherein the electrode surface is formed on a portion in the circumferential direction of the lead portion.

The defibrillation electrode according to a twentieth aspect of the present invention may also be provided with a stopper that is provided on at least one of the electrode portion and the lead portion, and whose maximum dimension in the radial direction of the lead portion is larger than the electrode portion and lead portion.

The stopper may have an asymmetrical shape when viewed from the axial direction of the lead portion.

In the defibrillation electrode according to a twenty-first aspect of the present invention, the implantable defibrillation system according to the twentieth aspect includes: the defibrillation electrode of the present invention; an implantable defibrillator that is connected to the connector; an introduction component that is able to have the defibrillation electrode either inserted inside it or mounted running alongside it, and whose distal end side is able to bluntly dissect biological tissue; and an observation device that observes an area in front of the introduction component.

### BRIEF DESCRIPTION OF THE DRAWINGS

FIG. 1 is a view showing the overall structure of a nerve stimulation electrode implantation system according to a first embodiment of the present invention.

FIG. 2 is a view showing a partial cross-section of a distal end side of the nerve stimulation electrode implantation system.

FIG. 3 is a frontal view showing the distal end side of the nerve stimulation electrode implantation system.

FIG. 4 is an enlarged view showing a distal end side of a peeling portion of the nerve stimulation electrode implantation system.

FIG. 5 is a view showing a partial cross-section of a nerve stimulation electrode through which a stylet is inserted.

FIG. 6 is a cross-sectional view taken along a line A-A shown in FIG. 5.

FIG. 7 is a view showing another example of the shape of a supporting portion of the nerve stimulation electrode.

FIG. 8 is a view showing an operation when the nerve stimulation electrode implantation system is being used.

FIG. 9 is a view showing an access path of the nerve stimulation electrode implantation system and the location of peripheral organs.

FIG. 10A is a view showing peeling processing performed by the peeling portion.

FIG. 10B is a view showing peeling processing performed by the peeling portion.

FIG. 11 is a view showing an operation when the nerve stimulation electrode is implanted.

FIG. 12 is a view showing an operation when the nerve stimulation electrode is implanted.

FIG. 13 is a view showing a partial cross-section of a distal end side of a modified example of the nerve stimulation electrode implantation system.

FIG. 14 is a frontal view showing the distal end side of the nerve stimulation electrode implantation system.

FIG. 15 is a view showing the distal end side of a nerve stimulation electrode implantation system according to a second embodiment of the present invention.

FIG. 16 is a frontal view showing the distal end side of a nerve stimulation electrode implantation system according to a second embodiment of the present invention.

FIG. 17 is a view showing the distal end side of a nerve stimulation electrode implantation system according to a third embodiment of the present invention.

FIG. 18 is a frontal view showing the distal end side of a nerve stimulation electrode implantation system according to a third embodiment of the present invention.

FIG. 19 is a view showing one method of use of the nerve stimulation electrode implantation system.

FIG. 20 is a view as seen from the front of the method of use of the nerve stimulation electrode implantation system.

FIG. 21 is a view showing another example of a nerve stimulation electrode.

FIG. 22A is a view showing an operation when the nerve stimulation electrode is implanted.

FIG. 22B is a view showing an operation when the nerve stimulation electrode is implanted.

FIG. 22C is a view showing an operation when the nerve stimulation electrode is implanted

FIG. 23 is a view showing another example of a nerve stimulation electrode.

FIG. 24 is a view showing the nerve stimulation electrode in a natural state.

FIG. 25 is a view showing a state in which the nerve stimulation electrode is fitted to a peeling portion.

FIG. 26 is a view showing an operation when the nerve stimulation electrode is implanted.

FIG. 27 is a view showing an operation when the nerve stimulation electrode is implanted.

FIG. 28 is a view showing an operation when the nerve stimulation electrode is implanted.

FIG. 29 is a view showing an operation when the nerve stimulation electrode is implanted.

FIG. 30 is a view showing the overall structure of a nerve stimulation electrode implantation system according to a fourth embodiment of the present invention.

FIG. 31 is a view showing a partial cross-section of a distal end side of the nerve stimulation electrode implantation system.

FIG. 32 is a frontal view of the distal end side of the nerve stimulation electrode implantation system.

FIG. 33 is an enlarged view showing a distal end side of a peeling portion.

FIG. 34 is a view showing a partial cross-section of a nerve stimulation electrode through which a stylet is inserted.

FIG. 35 is a cross-sectional view taken along a line A-A shown in FIG. 34.

FIG. 36 is a view showing an operation when the nerve stimulation electrode implantation system is being used.

FIG. 37 is a view showing an access path of the nerve stimulation electrode implantation system and the location of peripheral organs.

FIG. 38A is a view showing peeling processing performed by the peeling portion.

FIG. 38B is a view showing peeling processing performed by the peeling portion.

FIG. 39 is a view showing an operation when the nerve stimulation electrode is implanted.

FIG. 40 is a view showing an operation when the nerve stimulation electrode is implanted.

FIG. 41 is a side view showing main section of a nerve stimulation electrode implantation system of a modified example of the fourth embodiment of the present invention.

FIG. 42 is a side view showing principal portions of a nerve stimulation electrode implantation system of a modified example of the fourth embodiment of the present invention.

FIG. 43 is a side view showing main section of a nerve stimulation electrode implantation system of a modified example of the fourth embodiment of the present invention.

FIG. 44 is a view showing the overall structure of a nerve stimulation electrode implantation system according to a fifth embodiment of the present invention.

FIG. 45 is a view showing the overall structure of a nerve stimulation electrode implantation system according to a sixth embodiment of the present invention.

FIG. 46 is a view showing an attachment position of a defibrillation electrode according to a seventh embodiment of the present invention.

FIG. 47 is a view showing a different cross-section of the attachment position of the defibrillation electrode.

FIG. 48 is a view showing a different cross-section of the attachment position of the defibrillation electrode.

FIG. 49 is a frontal view of the defibrillation electrode.

FIG. 50 is a left-side view of the defibrillation electrode.

FIG. 51 is a cross-sectional view of a lead wire of a lead portion of the defibrillation electrode.

FIG. 52 is a cross-sectional view of a connecting portion between a lead portion and an electrode portion.

FIG. 53 is a view showing a preparatory state for an implantation surgical procedure for the defibrillation electrode.

FIG. 54 is a view showing one action of this implantation surgical procedure.

FIG. 55 is a view showing an introduction passage of each defibrillation electrode of the implantation surgical procedure.

FIG. 56 is a view showing one action of the implantation surgical procedure.

FIG. 57 is a view showing another example of an index mark.

FIG. 58 is a view showing another example of an index mark.

FIG. 59 is a frontal view of a defibrillation electrode according to an eighth embodiment of the present invention.

FIG. 60 is a left-side view of the defibrillation electrode.

FIG. 61 is a frontal view showing a modified example of the defibrillation electrode.

FIG. 62 is a frontal view showing the modified example.

FIG. 63 is an enlarged view of the distal end side of the defibrillation electrode of the modified example which is provided with another stopper.

FIG. 64 is an enlarged view as seen from a different angle of the distal end side of the defibrillation electrode.

FIG. 65 is an enlarged view of the distal end side of the defibrillation electrode of the modified example which is provided with another stopper.

FIG. 66 shows a front view and a bottom view of a defibrillation electrode according to a ninth embodiment of the present invention.

FIG. 67 is a left-side view of the defibrillation electrode.

FIG. 68 is a view showing an endoscope of an implantable defibrillation system of a modified example according to any of a seventh through ninth embodiment of the present invention.

FIG. 69 is a cross-sectional view showing a distal end side of the endoscope.

FIG. 70 is a view showing another example of the endoscope of the implantable defibrillation system of the modified example according to any of a seventh through ninth embodiment of the present invention.

FIG. 71 is a view showing another example of the endoscope of the implantable defibrillation system of the modified example according to any of a seventh through ninth embodiment of the present invention.

FIG. 72 is a cross-sectional view showing the distal end side of the endoscope.

FIG. 73 is a view showing another example of the endoscope of the implantable defibrillation system of the modified example according to any of a seventh through ninth embodiment of the present invention.

FIG. 74 is an enlarged view showing the distal end side of the endoscope.

FIG. 75 is a view showing a holder which is used in the implantable defibrillation system.

FIG. 76 is a view showing an example in which the holder and cap are attached to a normal endoscope.

FIG. 77 is a view showing another example of the implantation position of a defibrillation electrode.

FIG. 78 is a view showing another example of the implantation position of a defibrillation electrode.

FIG. 79 is a view showing another example of the implantation position of a defibrillation electrode.

FIG. 80 is a view showing an introduction passage for the defibrillation electrode of the example.

### DETAILED DESCRIPTION OF THE INVENTION

### First Embodiment

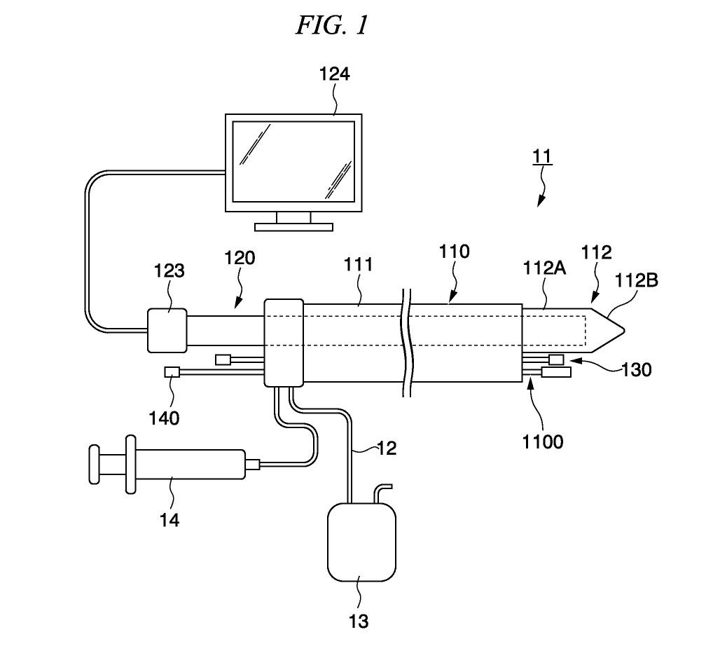

A first embodiment of the present invention will now be described with reference made to FIG. 1 through FIG. 14.

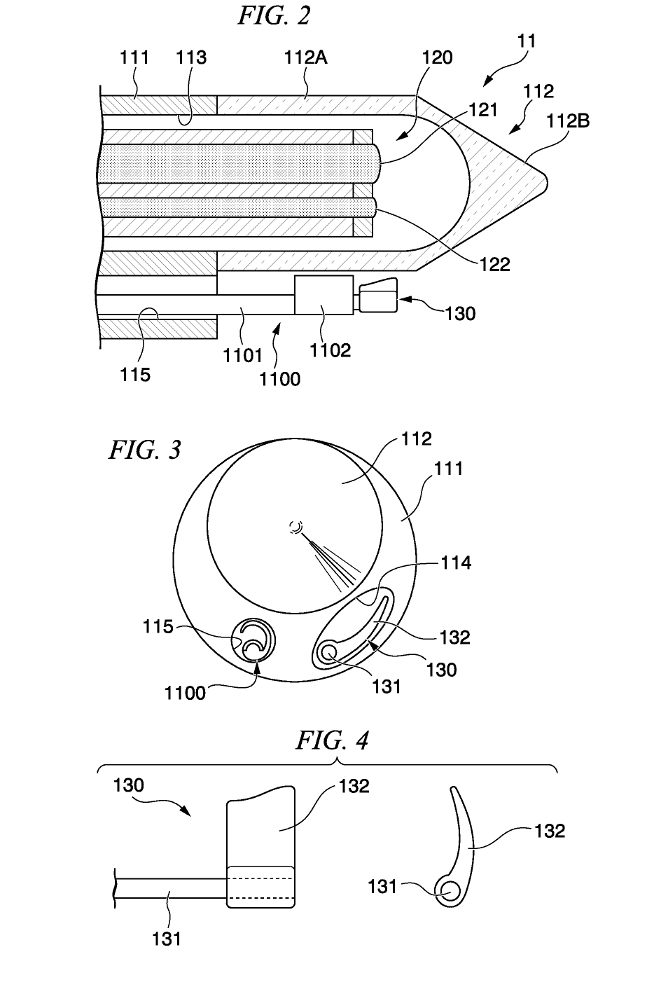

FIG. 1 is a view showing the overall structure of a nerve stimulation electrode implantation system (hereinafter, this will be referred to simply as an implantation system) 11 of the present embodiment. FIG. 2 shows a partial cross-section of the distal end side of the implantation system 11, while FIG. 3 is a frontal view of the distal end side of the implantation system 11.

The implantation system 11 performs various treatments in order to introduce and implant a nerve stimulation electrode (hereinafter, this will be referred to simply as an electrode) 1100 in intended nerve tissue such as a vagus nerve or the like by passing it through the inside of tissue such as connective tissue and the like. The implantation system 11 is provided with a substantially cylinder-shaped introduction portion 110, an observation portion 120 that is inserted in the introduction portion 110, a peeling portion 130 that is used to perform treatment in order for the electrode 1100 to be implanted in the subject nerve tissue, and a stylet (i.e., an electrode operating component) 140 that is used to perform the implantation operation of the nerve stimulation electrode 1100.

The introduction portion 110 is provided with a substantially circular cylinder-shaped main body 111, and with a transparent cap (i.e., incision component) 112 that is attached to a distal end portion of the main body 111. As is shown in FIG. 2 and FIG. 3, the main body 111 has three lumens (i.e., internal cavities), namely, a first lumen (i.e., a first internal cavity) 113 in which the observation portion 120 is inserted, a second lumen (i.e., a second internal cavity) 114 in which the peeling portion 130 is inserted, and a third lumen (i.e., an electrode cavity) 115 in which the electrode 1100 and the stylet 140 are inserted. A commonly known multi-lumen tube or the like can be used for the main body 111. The material used to make the main body 111 is not particularly restricted as long as it has sufficient rigidity to enable it to incise surrounding tissue (described below) and be moved forwards and backwards, and a metal such as stainless steel, or a resin or the like can be favorably used. From the standpoint of simplifying the operation to introduce the introduction portion 110, it is preferable for the outer diameter of the introduction portion 110 to be less than or equal to 6 millimeters (mm).

The cap 112 is a transparent component formed from resin or the like, and is attached to the distal end portion of the main body 111 so as to only seals off the first lumen 113. The cap 112 may be provided with a intended coloring as long as it still has sufficient transparency to enable the periphery of the cap 112 to be observed from the inserted observation portion 120. The cap 112 has a circular cylinder portion 112A on the proximal end side thereof which is connected to the main body 111, and a conical portion (i.e., a blunt dissection portion) 112B which is located further to the distal end side than the circular cylinder portion 112A. The observation portion 120 which is inserted inside the first lumen 113 is able to be moved forwards and backwards through the internal cavity of the circular cylinder portion 112A, and is also able to be rotated around its own axis relative to the introduction portion 110. The surface on the distal end side of the internal cavity of the circular cylinder portion 112A is formed so as to have a predetermined curvature in order to improve the performance of the observation portion 120. The surface on the distal end side of the internal cavity of the circular cylinder portion 112A may be provided with an optical coating film may be formed thereon such that it does not obstruct the observing of illumination light (described below) of the observation portion 120.

The conical portion 112B has a radius of curvature on the distal end thereof which is set to, for example, approximately 0.2 mm, and has a blunt distal end. By directly pressing the insertion portion 110 with the conical portion 112B located in the forefront, it is possible to introduce the implantation system 11 into the vicinity of the subject tissue while bluntly incising the surrounding tissue located around the cap 112. This operation is described in further detail below in the description of the surgical procedure.

The observation portion 120 has an outer diameter that enables it to be inserted through the inside of the first lumen 113. Moreover, as is shown in FIG. 2, the observation portion 120 has an observation optical system 121, and an illumination device 122 such as a light guide or the like that illuminates the field of view of the observation optical system 121, and these are both provided at the distal end portion of the observation portion 120. As is shown in FIG. 1, the observation portion 120 is connected to an imaging device 123 at the proximal end thereof by means of a commonly known image guide or the like. As a result, the observation portion 120 is able to obtain video images within the field of view of the observation optical system 121. The imaging device 123 is connected to a monitor 124, and video images obtained by the imaging device 123 are displayed on the monitor 124.

If the illumination device 122 is a light guide, then it is connected to a light source (not shown) which is provided within the imaging device 123. Instead of a light guide, it is also possible to mount a light-emitting component such as an LED or the like at the distal end as an illumination device.

A known endoscope device can be favorably used as the observation portion 120 provided that the dimensions of the outer diameter and the like thereof are suitable values.

FIG. 4 is an enlarged view of the distal end side of the peeling portion 130. As is shown in FIG. 3 and FIG. 4, the peeling portion 130 has an elongated rod 131, and a spatula portion 132 which is attached to a distal end portion of the rod 131. The material used to make the rod 131 is not particularly restricted as long as it has sufficient rigidity to enable it to be moved forwards and backwards in an axial direction, and a metal such as stainless steel, or a resin or the like can be favorably used.

The spatula portion 132 is formed in a plate shape or a sheet shape, and is attached such that it extends in a direction which is substantially orthogonal to the axial direction of the rod 131. The spatula portion 132 has sufficient rigidity to enable it to peel off connective tissue and the like which surrounds the nerve tissue (hereinafter, this will be referred to as surrounding tissue). Moreover, the spatula portion 132 can be formed from the same type of material as the rod 131. A peripheral edge on the distal end side of the spatula portion 132 which extends out from the rod 131 is able to bluntly perform a peeling operation (described below). As a result, the peripheral edge on the distal end side of the spatula portion 132 is formed so as to be blunt in the same way as the distal end of the conical portion 112B.

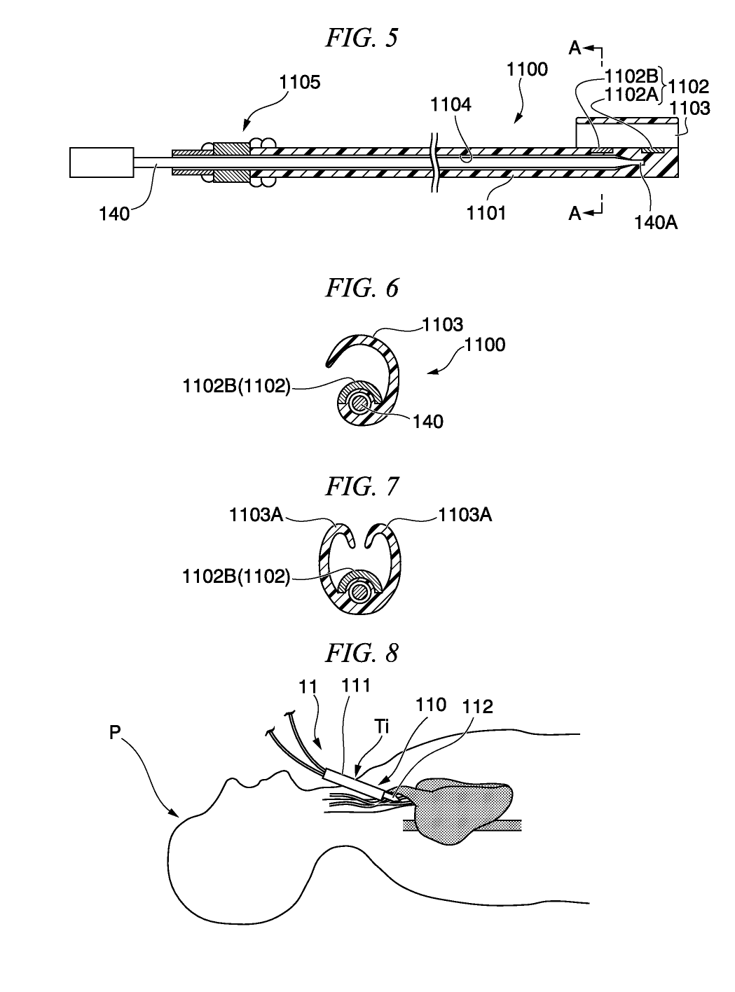

The structures of the stylet 140 and the electrode 1100 will now be described. FIG. 5 shows a partial cross-section of the electrode 1100 into which the stylet 140 is inserted. FIG. 6 is a cross-sectional view taken along a line A-A in FIG. 5.

The electrode 1100 has an elongated lead portion 1101, an electrode portion 1102 which is provided at the distal end side of the lead portion 1101, and a supporting portion 1103 which supports the electrode portion 1102 such that it makes contact with nerve tissue.

The lead portion 1101 is formed in a substantially circular cylinder shape having an internal cavity 1104 from a material such as polyurethane or the like which is highly stable inside a living organism, and the stylet 140 is able to be inserted inside this internal cavity. The internal cavity 1104 is not open on the distal end side of the lead portion 1101. In addition, the distal end portion of the internal cavity 1104 is flattened in a radial direction. As a result of this, the minimum dimension in the radial direction of this distal end portion of the internal cavity 1104 is shorter than the dimension in the radial direction of the substantially circular column-shaped internal cavity 1104.

The lead portion 1101 has a plurality of leads (not shown). In each lead, conductive core wires are formed using highly durable MP35N wires or 35MLT wires. In addition, each lead is formed with a non-conductive coating made from an ETFE material coated on each of the conductive core wires, and these are then placed inside the internal cavity 1104. The distal end side of each lead is connected to the electrode portion 1102, while the proximal end side of each lead is connected to a connector 1105 which is used to connect to a nerve stimulator (not shown). A commonly known connector which matches the nerve stimulator to which it is connected can be suitably selected for the connector 1105. For example, an IS1 connector or the like can be used.

Note that in the above described structure, the leads may also be formed by embedding only the core wires (without non-conductive coating) inside the substantially circular cylinder-shaped peripheral wall of the lead portion 1101.

The structure of the electrode portion 1102 is not particularly restricted as long as it is able to impart electrical stimulus to nerve tissue with which it comes into contact. However, as is shown in FIG. 5, it is preferable for the electrode portion 1102 to be a bipolar type of electrode having two electrodes, namely, a negative electrode 1102A and a positive electrode 1102B. Platinum which is stable inside a living organism is used for the material for the negative electrode 1102A and the positive electrode 1102B. A titanium nitride (TiN) film having a micro-asperity structure is formed on the surfaces of the respective electrodes 1102A and 1102B so that there is reduced impedance with the surface of the living organism.

During implantation, only the side of the respective electrodes 1102A and 1102B that faces the nervous tissue is exposed. As a result, the surface of the respective electrodes 1102A and 1102B which is located on the opposite side is covered by silicone resin or the like, and is electrically insulated. Namely, any leakage of applied electrical energy to tissue or organs located peripherally to the nerve tissue is reduced.

If the subject nerve tissue is a vagus nerve, then it is common for the peeled-off nerve tissue to have an outer diameter of approximately 1-2 mm. Consequently, in order to fit in with the outer diameter of the peeled-off nerve tissue, it is preferable for the core wires of the lead and the electrodes of the electrode portion to be formed to not more than φ2 mm.

The supporting portion 1103 is formed by extending a portion of the silicone resin that covers a portion of the electrode portion 1102 in a substantially circular arc shape. In consideration of the function of the supporting portion 1103, it is preferable for this circular arc shape to be set such that the diameter of the circular arc is larger than the outer diameter of the nerve tissue. The supporting portion 1103 is elastically deformable, and supports nerve tissue by gripping it between itself and the electrode portion 1102. By doing this, the supporting portion 1103 is able to bring the respective electrodes 1102A and 1102B into contact with the nerve tissue. Because the shape of the supporting portion 1103 can be deformed so that it follows the outer circumferential surface of the electrode portion 1102, as is shown in FIG. 3, it is possible for the entire electrode 1100 to be housed inside the third lumen 115.

The number and configuration of the supporting portions may be suitably set in order to satisfy the aim of bringing the respective electrodes 1102A and 1102B into contact with nerve tissue. For example, as is shown in FIG. 7, it is possible to employ a structure in which a pair of supporting portions 1103A is provided such that inwardly curving sides thereof face each other, and such that nerve tissue is supported by being gripped between the pair of supporting portions 1103A. It is preferable for the thickness of the supporting portion to be less than or equal to 0.5 mm so that an excessive load is not applied to the subject tissue.

The stylet 140 is formed from a material such as resin or metal to dimensions that enable it to be inserted into the internal cavity 1104 of the lead portion 1101. A distal end portion 140A of the stylet 140 is flattened in a radial direction so as to correspond to the shape of the distal end portion of the internal cavity 1104. Accordingly, if the distal end portion 140A is inserted inside the distal end portion of the internal cavity 1104 and the stylet 140 is rotated, the electrode 1100 can be rotated around the axial direction of the lead portion 1101. In addition, by moving the stylet 140 forwards and backwards in the axial direction, and by pulling the proximal end side of the lead portion 1101 back towards an operator, the electrode portion 1102 can be made to protrude from or can be retracted back into the aperture at the distal end of the third lumen 115.

A watertight component such as an O-ring or the like (not shown) is attached to the proximal end side of the second lumen 114 and the third lumen 115. In addition, by inserting the peeling portion 130 and the electrode 1100 respectively into the second lumen 114 and the third lumen 115, the proximal end sides of the second lumen 114 and the third lumen 115 can be made watertight.

As is shown in FIG. 1, a drainage tube 12 is connected to the second lumen 114. A drainage pump 13 is connected to the drainage tube 12, and any body fluids or blood that have infiltrated the interior of the second lumen 114 from the aperture at the distal end of the second lumen 114 can be suctioned and discharged to the outside of the lumen. A syringe 14 is connected to the third lumen 115, and a physiological salt solution (i.e., saline) or the like can be supplied from the syringe 14 to the interior of the third lumen 115. As a result, saline can be released from the aperture at the distal end of the third lumen 115 so that the cap 12 can be washed, and the field of view of the observation portion 120 can be improved.

Operations when the implantation system 11 having the above described structure is put to use will now be described taking as an example a case in which the electrode 1100 is implanted in order to treat a right vagus nerve of a human (hereinafter, this will be referred to simply as a vagus nerve). When the nerve tissue adjacent to the heart which is to be stimulated is the vagus nerve, the passage from the body surface is easily distinguished from observation of the surrounding tissue. Moreover, the vagus nerve has the advantage that it is a short distance from the body surface and it is therefore easy to reach, so that access to it is comparatively easy.

A surgeon inserts the observation portion 120 into the first lumen 113 of the introduction portion 110, and the peeling portion 130 and the electrode 1100 in which the stylet 140 is inserted are inserted respectively into the second lumen 114 and the third lumen 115. By doing this, preparations before the implantation system 11 is put to use are completed. However, it is not necessarily that the peeling portion 130 and the electrode 1100 be inserted at this time. It is also possible for the peeling portion 130 and the electrode 1100 to be inserted after the introduction portion 110 has reached the vicinity of the vagus nerve. In this case, if necessary, it is preferable for stoppers to be fitted into the proximal end side of the second lumen 114 and the third lumen 115.

Next, the surgeon makes a small incision in the body surface of a patient P, and inserts the conical portion 112B of the introduction portion 110. In this example, as is shown in FIG. 8, the small incision is made adjacent to the superior thoracic aperture Ti of the patient P.

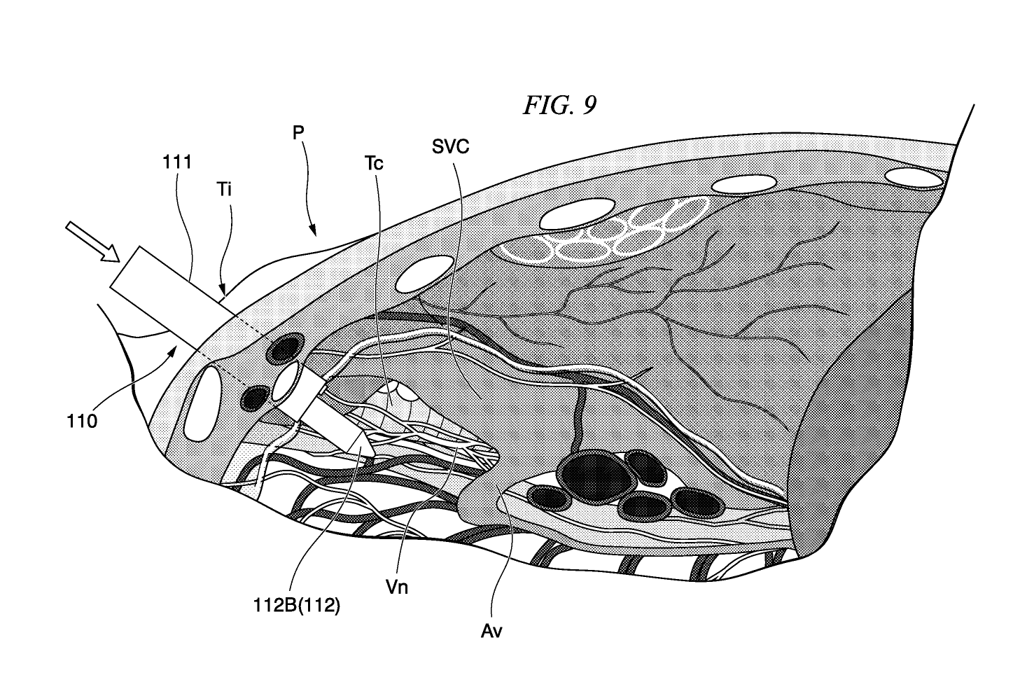

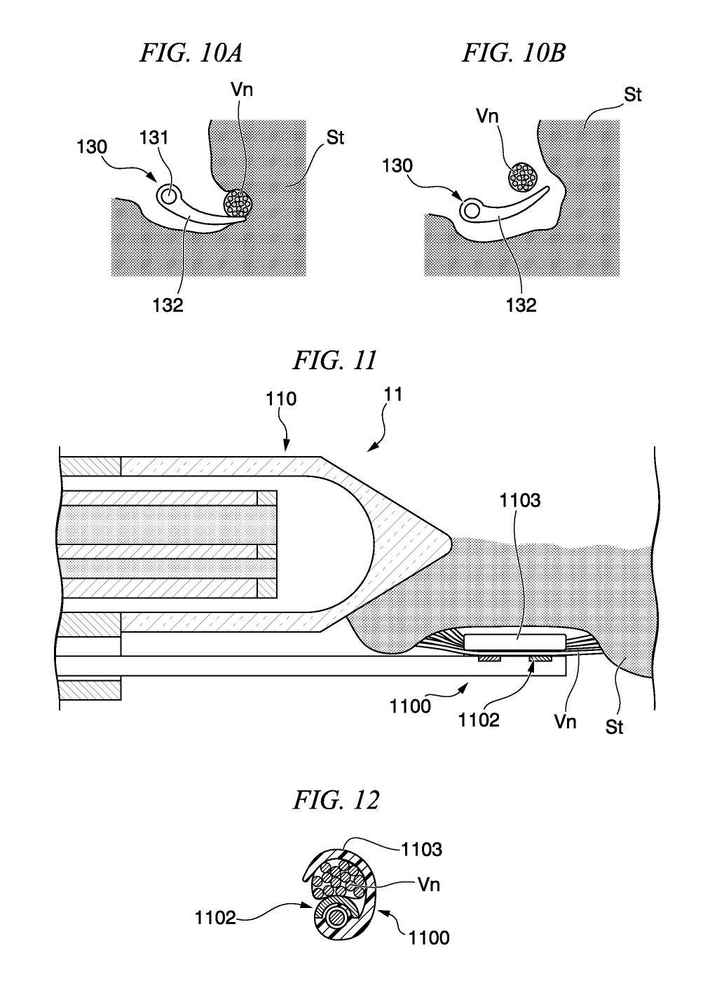

After the introduction portion 110 is inserted, the surgeon applies force in the axial direction while holding the main body 111 and while confirming the situation surrounding the cap 112 by means of the observation portion 120, and pushes the introduction portion 110 towards the body interior. As is shown in FIG. 9, because the superior thoracic aperture Ti is located close to the trachea Tc, when the introduction portion 110 is inserted, the white tube-shaped trachea Tc shortly becomes visible within the field of view of the observation portion 120. If the introduction portion 110 is moved forward along the trachea Tc, it easily reaches the vicinity of the vagus nerve Vn. Consequently, in this example, the trachea Tc can be used as a guide to the vagus nerve.

A large quantity of comparatively soft loose connective tissue is present around the introduction portion 110 in the access passage of the implantation system 11 in this example. Because of this, by pushing the introduction portion 110 forwards with the conical portion 112B in the forefront thereof, biological tissue such as the loose connective tissue and the like located in the front thereof is bluntly dissected by the conical portion 112B and the introduction portion 110 is able to move forward. Accordingly, it is not necessary to use a highly rigid member such as a metal pipe in the main body 111 when the introduction portion 110 is being moved forwards. Furthermore, a large amount of force is not necessary when the introduction portion 110 is being pushed in order to make it move forwards. Moreover, when the introduction portion 110 is advancing along the trachea Tc, because the boundary interface between the trachea Tc and the biological tissue around the trachea Tc is easily split, the blunt dissection can be made to proceed even more easily. Because the periphery of the trachea Tc is covered by cartilage, as long as the distal end of the conical portion 112B is blunt, there is no possibility that the trachea Tc will be harmed by the forward movement of the introduction portion 110.

There are few blood vessels in the loose connective tissue that occupies the major portion of the biological tissue, and there is also substantially no cutting of blood vessels by the blunt conical portion 112B. Consequently, although there is no particularly large amount of bleeding while the introduction portion 110 is being moved forwards, because bodily fluids such as interstitial fluids are extravasate from the bluntly dissected biological tissue, if, necessary, these can be suctioned and expelled using the drainage pump 13.

The observation portion 120 which is inserted into the introduction portion 110 is able to be moved forwards and backwards in an axial direction, and is also able to be rotated around its own axis. Accordingly, if a surgeon appropriately operates the observation portion 120 within the first lumen 113, that surgeon is able to obtain an excellent view of the area in front of the conical portion 112B and of the entire periphery of the cap 112 including the periphery of the circular cylinder portion 112A. Accordingly, it is possible, while confirming the biological tissue surrounding the cap 112, for a surgeon to make the distal end portion of the introduction portion 110 move forward easily as far as the vicinity of the heart where the vagus nerve Vn is located.

When the introduction portion 110 reaches the vicinity of the heart, the superior vena cava SVC and the azygos vein Av which is joined to the superior vena cava SVC become visible within the field of view of the observation portion 120. The vagus nerve Vn runs adjacent to the superior vena cava SVC and the azygos vein Av, and is verified as a white band-shaped tissue within the field of view of the observation portion 120. Accordingly, when the azygos vein Av is verified, by carefully observing the periphery of the cap 112 using the observation portion 120, it becomes possible to easily find the vagus nerve Vn using the azygos vein Av as an indicator. In the implantation system 11, the imaging device 123 and the monitor 124 are preferably constructed such that they are able to display color images as this makes it possible to easily determine tissue through variations in color.

When the vagus nerve Vn is verified, the surgeon removes a portion of the surrounding tissue so as to expose a portion of the vagus nerve Vn, and also peels this portion away from the surrounding tissue such that it is possible to implant the electrode 1100. The surgeon then causes the rod 131 of the peeling portion 130 which is inserted into the second lumen 114 to move forward and to protrude from the distal end of the introduction portion 110. If necessary, the surgeon may remove the surrounding tissue at the surface of the vagus nerve Vn using the spatula portion 132 while moving the introduction portion 110 forwards and backwards and in rotation. Because the composition of this surrounding tissue is substantially the same as that of the above-described biological tissue, it can easily be removed by the spatula 132. Thereafter, as is shown in FIG. 10A, by pushing and pulling the rod 131 while the end portion of the spatula portion 132 is pressed against the vagus nerve Vn, the surgeon is able to move the spatula portion 132 in the axial direction of the rod 131. As a result, as is shown in FIG. 10B, the vagus nerve Vn can be easily peeled away from the surrounding tissue St. After this peeling processing has ended, the surgeon re-houses the peeling portion 130 within the second lumen 114.

When the vagus nerve Vn peeling processing has ended, the surgeon causes the electrode 1100 inside which the stylet 140 is inserted to protrude from the third lumen 115. While adjusting the position of the supporting portion 1103 by rotating the stylet 140, the surgeon then inserts the free end of the supporting portion 1103 between the vagus nerve Vn and the surrounding tissue St such that it catches on the peeled off portion of the vagus nerve Vn. As a result, as is shown in FIG. 11 and FIG. 12, the vagus nerve Vn is supported between the supporting portion 1103 and the electrode portion 1102, and the electrode 1100 is then left implanted in the vagus nerve Vn such that the electrode portion 1102 and the vagus nerve Vn are firmly stuck together.

Note that during the peeling processing for the vagus nerve Vn and when the electrode 1100 is being implanted and so on, the field of view of the observation portion 120 can be made clearer if a physiological salt solution (i.e., saline) is injected into or suctioned from the space where this treatment is being performed using the syringe 14. It is also possible to supply carbon dioxide gas to the relevant space instead of saline, and to form a space where treatment may be performed by pushing out the surrounding tissue.

After the electrode 1100 is implanted, the surgeon extracts the stylet 140 from the electrode 1100, and withdraws the introduction portion 110 to the outside of the body by pulling it backwards. Originally, the surrounding tissue and biological tissue completely filled the access path by which the introduction portion 110 entered the body. Because of this, as the introduction portion 110 is withdrawn, the path taken by the introduction portion becomes completely filled up again by the surrounding tissue and biological tissue. Accordingly, after the introduction portion 110 is withdrawn, the area surrounding the implanted electrode 1100 is also substantially filled up by the surrounding tissue and biological tissue, and the electrode 1100 becomes held in the implantation position by this surrounding tissue and biological tissue. Consequently, there is no need to provide any fixing sutures or the like after the electrode 1100 is implanted. Moreover, because there is little movement in the vicinity of the superior thoracic aperture Ti which is caused by physical activity by the patient, the electrode position is stable.

By withdrawing the introduction portion 110, the series of tasks which are performed in order to implant the electrode 1100 is ended.

After the electrode 1100 is implanted, the connector 1105 of the electrode 1100 is connected to the nerve stimulator, and treatment by means of electrical stimulation is started. For example, a rectangular pulse voltage having a width of between several tens of microseconds (μsec) and several milliseconds (msec) is applied at a frequency of several tens of Hertz (Hz). The voltage value of the rectangular pulse voltage is appropriately set between several volts and several tens of volts. Depending on the treatment content, either continuous stimulation or intermittent stimulation may be selected, and the period for which the electrical stimulation is performed is suitably determined depending on the treatment.

After the treatment is ended, the connector 1105 is removed from the nerve stimulator, and the supporting portion 1103 is moved away from the vagus nerve Vn by pulling the end portion of the lead portion 1101. By further pulling on the center portion, the electrode 1100 can be extracted through the surrounding tissue and biological tissue to the outside of the body. Namely, because there is no need to perform any comparatively highly invasive surgical treatments in order to remove the electrode 1100, the load on the patient when the electrode is removed is considerably alleviated.

As has been described above, the implantation system 11 of the present embodiment is provided with the introduction portion 110 which is equipped with the cap 112 having the distal end portion 112A, the observation portion 120, and the peeling portion 130 which is used to remove surrounding tissue from around the nerve tissue. Accordingly, by bluntly incising biological tissue using the distal end portion 112A while at the same time using the observation portion to verify the area surrounding the cap 112 which is placed at the distal end of the introduction portion 110, it is possible for the vicinity of the subject nerve tissue to be reached easily and with a low level of invasiveness. In addition, by performing peeling treatment on the nerve tissue using the peeling portion 130, it is possible to remove a portion of the surrounding tissue from this tissue, and thus create a state in which the electrode 1100 can be easily implanted.

Accordingly, if the subject tissue is the vagus nerve Vn, compared with a method in which the pleura is pierced by a trocar or the like and is accessed from inside the thoracic cavity, or a method in which a blood vessel is incised and the electrode is implanted in the blood vessel wall, not only is it possible to implant an electrode in a quicker time and with less invasiveness, but the electrode can be made to contact the nerve tissue directly so that nerve stimulation treatment can be performed with a high degree of efficiency.

Furthermore, because the electrode is implanted in nerve tissue which is located closer to the heart, it is possible to reduce any stimulus to tissue and organs other than the heart which are not subject to treatment, and throat blockages and coughing and the like can be suppressed.

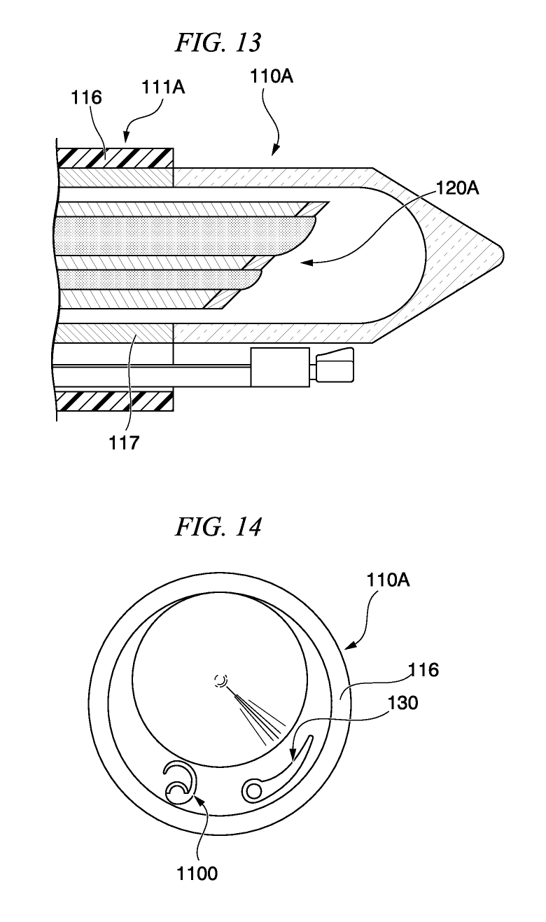

In the present embodiment, a description is given of an example in which the introduction portion has three internal cavities, namely, the first and second lumens, and a third lumen which is the cavity for the electrode, however, instead of this, as is shown in FIG. 13 and FIG. 14, an introduction portion 110A is constructed using a main body 111A in which an inner tube 117 is inserted and then fixed inside an outer tube 116. In this case, the observation portion 120 is inserted into the internal cavity of the inner tube 117, and the space between the outer circumferential surface of the inner tube 117 and the inner circumferential surface of the outer tube 116 doubles as both a second internal cavity and an electrode cavity, and the peeling portion 130 and the electrode 1100 inside which the stylet 140 is inserted can be housed therein.

Moreover, during a surgical procedure, in order to obtain a better field of view, it is also possible, if necessary as is shown in FIG. 13, for an oblique-view type of observation portion 120A whose direction of observation is inclined relative to the axis to be used. Furthermore, a side-view type of observation portion having observation windows in a side surface thereof may also be used in combination with the observation portion.

### Second Embodiment

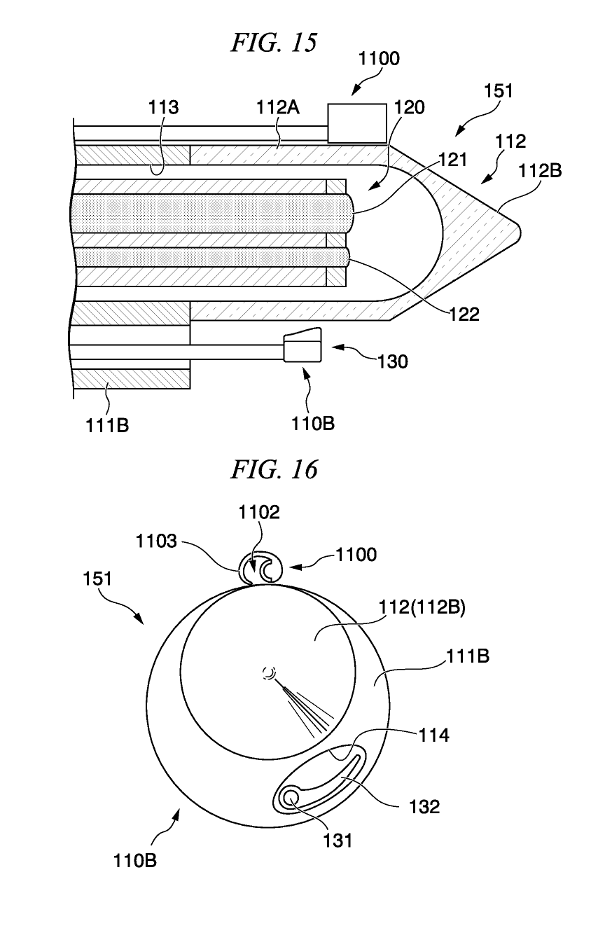

Next, a second embodiment of the present invention will be described with reference made to FIG. 15 and FIG. 16. An implantation system 151 of the present embodiment differs from the above described implantation system 11 in that the electrode is introduced to subject tissue without being inserted inside an introduction portion. Note that in the following description, structural elements that are the same as those already described are given the same symbols, and any duplicated description thereof is omitted.

FIG. 15 shows a partial cross-section of a distal end side of the implantation system 151, while FIG. 16 is a frontal view of this same distal end side. As is shown in FIG. 15 and FIG. 16, a main body 111B of an introduction portion 110B is not provided with a third lumen.

When the electrode 1100 is to be implanted in the vagus nerve Vn using the implantation system 151, firstly, without the electrode 1100 and the stylet 140 being fitted in position, the procedure is performed in the same way as in the first embodiment as far as the vagus nerve Vn peeling processing. When a portion of the vagus nerve Vn is peeled away from the surrounding tissue, a surgeon inserts the electrode 1100 inside which the stylet 140 is inserted to a position where the introduction portion 110B has already been inserted, and makes the electrode 1100 move forward along the outer circumferential surface of the introduction portion 110B. At this time, it is preferable for the supporting portion 1103 to be extended and for the electrode 1100 to be moved forward after it has first been positioned against the outer circumferential surface of the introduction portion 110B, as this enables it to advance more reliably alongside the introduction portion 110 B.

When the electrode portion 1102 has reached the vicinity of the distal end of the introduction portion 110B, the surgeon performs the same type of operation as in the first embodiment so as to implant the electrode 1100 in the vagus nerve Vn. In order to secure a working space for the implantation of the electrode 1100, it is preferable for the introduction portion 110B to first be moved forward slightly in front of the position where the nerve tissue is located, and to then be pulled back to the position of the nerve tissue. If this method is employed, then also in cases when the supporting portion 1103 is moved forward along the outer circumferential surface of the introduction portion 110B, by positioning the electrode portion 1102 in front of the distal end of the introduction portion 110B, the supporting portion 1103 can easily be restored to a shape that enables it to engage with the vagus nerve Vn. At this time, if carbon dioxide gas or a fluid such as saline is supplied from the second lumen 114, it is possible to secure a larger working space.

In the implantation system 151 of the present embodiment as well, in the same way as in the implantation system 11, it is possible to implant the electrode 1100 in a short time and with a low level of invasiveness. In addition, it is possible to reduce any stimulus to tissue and organs other than the heart which are not subject to treatment. Although the electrode 1100 into which the stylet 140 has been inserted is introduced to the vicinity of nerve tissue without passing through the inside of the introduction portion 110B, because it is introduced through a tunnel previously formed within the biological tissue by the introduction portion 110B while using the outer circumferential surface of the introduction portion 110 as a guide, it is able to easily and reliably reach the subject nerve tissue.

Moreover, because the third lumen is not formed in the introduction portion 110B, the diameter of the introduction portion can be made even smaller. Note that it is also possible to provide the third lumen in the same way as the introduction portion 110, and to use this as a dedicated lumen for supplying air or fluid or for suctioning.

### Third Embodiment

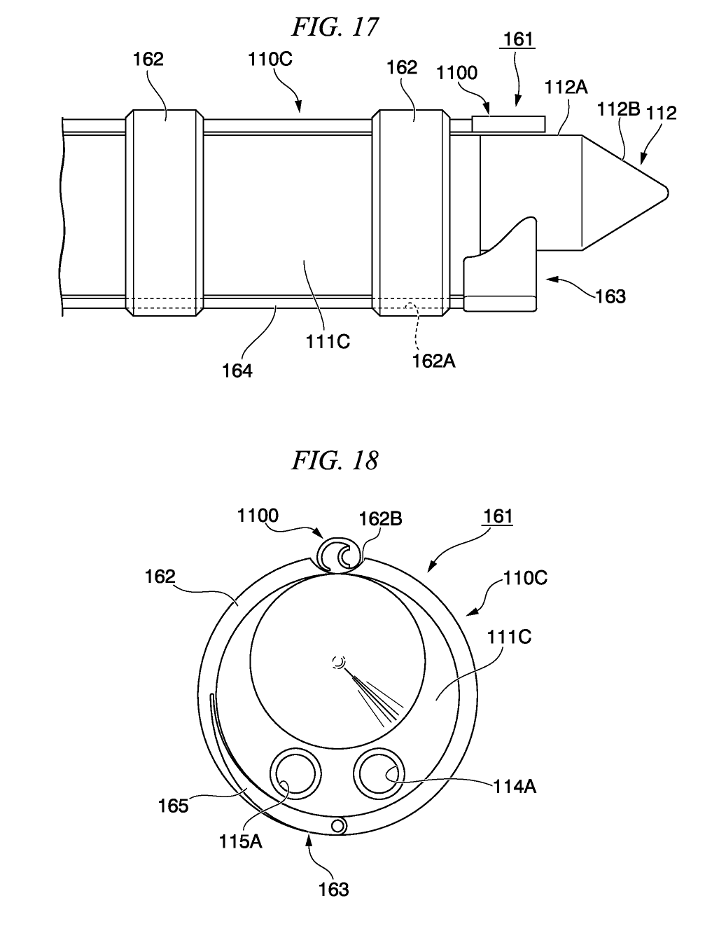

Next, a third embodiment of the present invention will be described with reference made to FIG. 17 through FIG. 20. An implantation system 161 of the present embodiment differs from the implantation systems of each of the above described embodiments in that the peeling portion is not inserted in the introduction portion.

FIG. 17 shows a distal end side of the implantation system 161. FIG. 18 is a frontal view of the distal end side of the implantation system 161. The peeling portion and the electrode are not inserted in a second lumen 114A and a third lumen 115A which are provided in an introduction portion 110C and, instead, these are used respectively as an air and fluid feed lumen and as a suctioning lumen.

A plurality of toroidal guides components 162 are attached to the outer circumference of a main body 111C of the introduction portion 110C with a predetermined distance between them. Each of the guide components 162 has an insertion aperture 162A which penetrates it in an axial direction, and a trench-shaped guide groove 162 B which is formed in the outer circumferential surface extending in the axial direction. In addition, the insertion aperture 162A and the guide groove 162B are fixed to the main body 111C such that each one of these is arranged on a straight line. Both end portions in the axial direction of each guide component 162 is tapered so that any increase in resistance when the introduction portion 110C is moved forwards or backwards inside a body is inhibited.

The basic structure of a peeling portion 163 is the same as that of the peeling portion 130, however, the dimensions of the rod 164 are such that it can be inserted into the insertion aperture 162A, and the radius of curvature of a spatula portion 165 when it is viewed from the axial direction of the peeling portion 163 is set to be substantially the same as that of the outer circumference of the main body 111C.

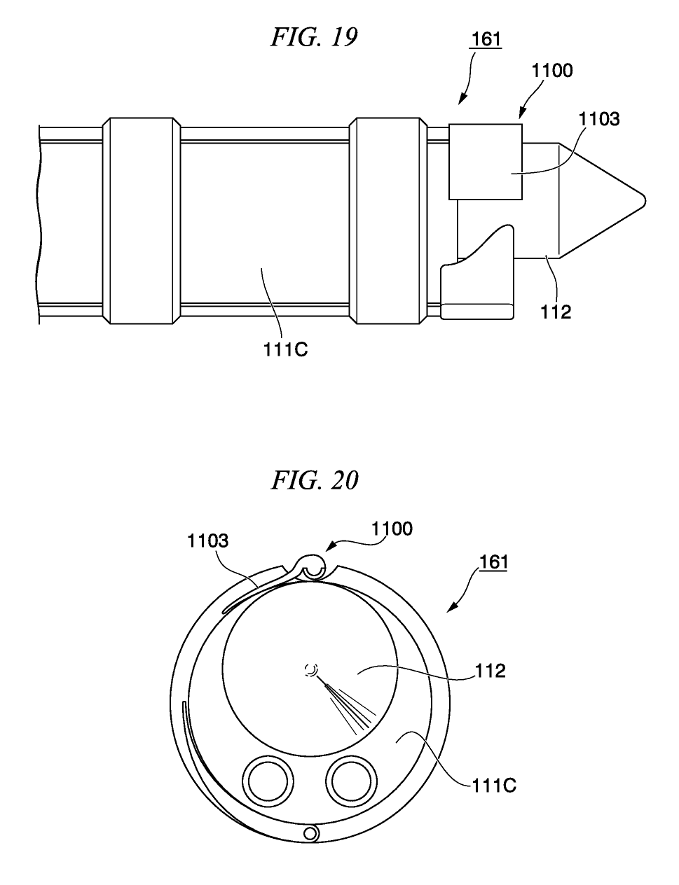

When the implantation system 161 is put to use, a proximal end of the rod 164 of the peeling portion 163 is inserted into the insertion aperture 162A of the guide component 162 which is located closest to the distal end side through to the insertion aperture 162A of each guide component 162, and the peeling portion 163 is mounted in the introduction portion 110C. It is also possible for the electrode 1100 to be mounted in the introduction portion 110C before the introduction portion 110C is inserted inside a body. It is also possible for the electrode 1100 to be inserted inside the body afterwards, as is the case in the second embodiment. In either case, the electrode 1100 is arranged so that it follows the guide groove 162B and can move forwards or backwards along this groove. If the electrode 1100 is mounted before the insertion inside the body, then as is shown in FIG. 19 and FIG. 20, the shape of the supporting portion 1103 may be adapted to conform to the outer circumferential surface of the main body 111C and the cap 112.

Inside the body, operations to move the peeling portion 163 forwards or backwards or to rotate it using the insertion aperture 162A of the guide component 162 as a guide are performed, and operations to move the electrode 1100 inside which the stylet 140 is inserted forwards or backwards or to rotate it using the guide groove 162B as a guide are also performed. The rest of the procedure is the same as in each of the above described embodiments.

In the implantation system 161 of the present embodiment as well, in the same way as in each of the first and the second embodiments of the present invention previously described, it is possible to implant the electrode 1100 in a short time and with a low level of invasiveness. In addition, it is possible to reduce any stimulus to tissue and organs other than the heart which are not subject to treatment.

The first through third embodiments of the present invention have been described above, however, the range of technology of the present invention is not limited to the above described embodiments and it is also possible for the component elements to be used in a variety of combinations, or for various modifications to be made to the respective component elements, or for various component elements to be omitted insofar as none of these actions has any effect on the spirit or scope of the present invention.

For example, in addition to the above described nerve stimulation electrode, it is also possible for nerve stimulation electrodes of various designs to be used as the nerve stimulation electrode which is implanted using the implantation system.

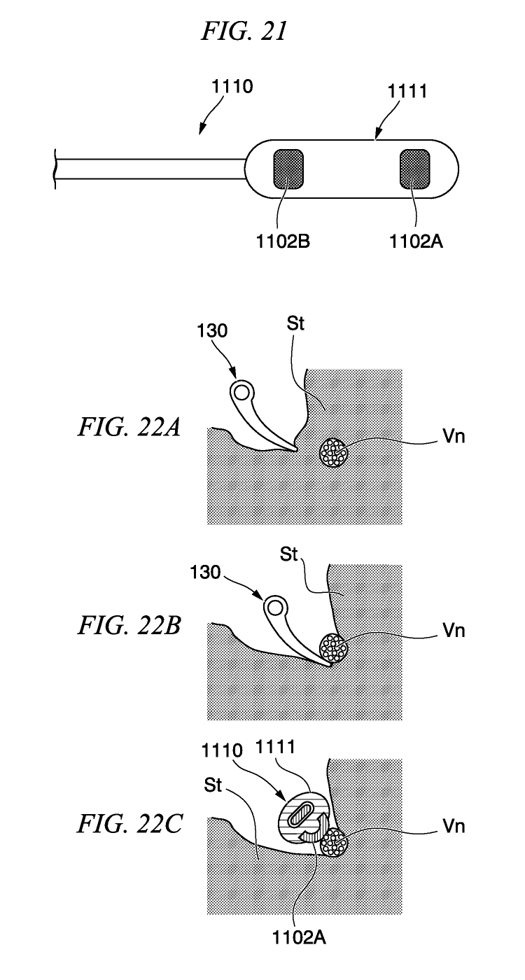

FIG. 21 shows a modified example of an electrode that can be implanted using the implantation system. This electrode 1110 has a structure in which no supporting portion is provided. An electrode portion 1111 is formed such that the cross-section thereof which is orthogonal to the axial direction of the lead portion 1101 has an elliptical shape (see FIG. 22C), and two electrodes, namely, a negative electrode 1102A and a positive electrode 1102B are formed on one surface in the short radius direction of the ellipse. The dimensions in the longitudinal direction, the transverse direction, and the thickness direction of the electrode portion 1111 are set, for example, to 6 mm, 3 mm, and 1.5 mm respectively. The distance in the longitudinal direction between the negative electrode 1102A and the positive electrode 1102B is set, for example, to 4 mm.

Because the electrode 1110 is not provided with the supporting portion, in the vagus nerve Vn peeling processing, as is shown in FIG. 22A and FIG. 22B, a sufficient portion of the surrounding tissue St is removed by the peeling portion 130 or the like so as to allow the electrode 1110 to be implanted, and so as to expose a portion of the vagus nerve Vn. Because the electrode 1110 is not attached to the vagus nerve Vn and is only pressed against it, it is not essential for the vagus nerve Vn to be completely peeled away from the surrounding tissue St.

After the peeling processing for the vagus nerve Vn, as is shown in FIG. 22C, the surgeon implants the electrode 1110 such that the electrodes 1102A and 1102B are in contact with the vagus nerve Vn. After the implantation, when the introduction portion is extracted, the area around the electrode portion 1111 is properly supported by the surrounding tissue St, and the state of contact between the electrode portion 1111 and the vagus nerve Vn is maintained.

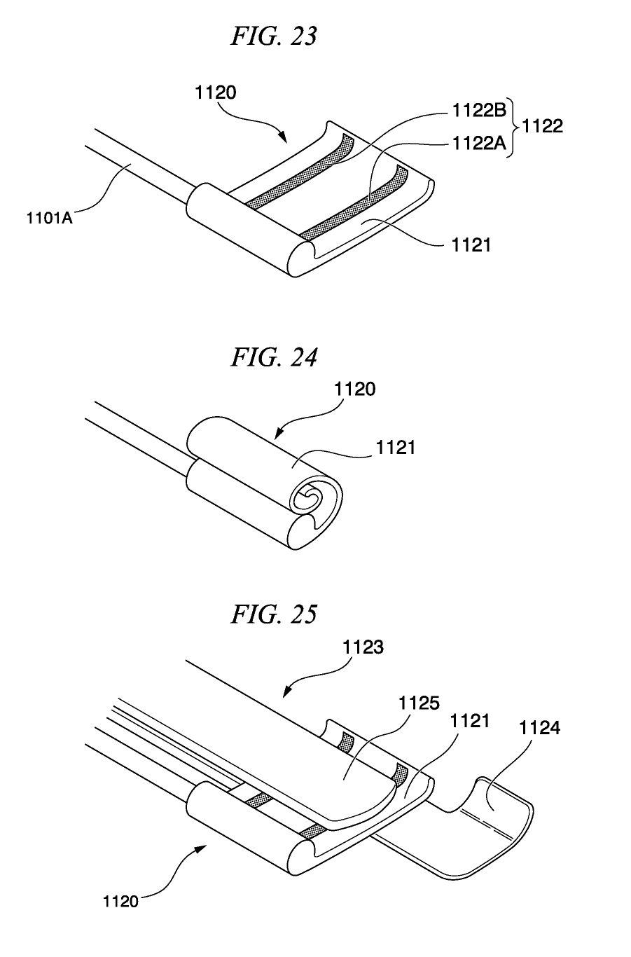

FIG. 23 and FIG. 24 show another modified example of an electrode. This electrode 1120 is provided with a supporting portion 1121 that is formed from silicone or the like, and with electrode portions 1122 that are formed on one surface of the supporting portion 1121. As is shown in FIG. 23, the supporting portion 1121 can be unrolled so as to be substantially flat, however, in its natural state without the application of any external force, as is shown in FIG. 24, it is curled at a predetermined curvature. Accordingly, it can be implanted by being suitably wound around linear tissue such as a nerve or the like. The negative electrode 1122A and the positive electrode 1122B of the electrode portion 1122 are each formed from platinum wire. In addition, the negative electrode 1122A and the positive electrode 1122B of the electrode portion 1122 are positioned substantially parallel with the uncurling direction (i.e., a direction in which the supporting portion 1121 extends from the lead portion 1101A when the supporting portion 1121 is unwound so as to be substantially flat) of the supporting portion 1121 on the surface which is on the inner side of the curvature of the supporting portion 1121.

The lead portion 1101A does not have an internal cavity so that the stylet 140 cannot be inserted therein to.

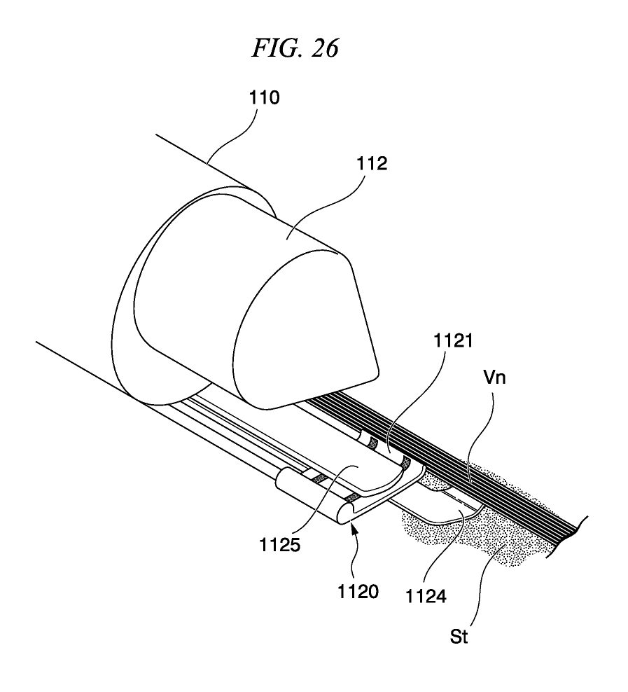

FIG. 25 shows an example of a peeling portion 1123 that is used together with the electrode 1120. The peeling portion 1123 is formed substantially in a trench shape, and a cross-section which is orthogonal to the longitudinal direction thereof has a circular arc shape (i.e., a substantially circular arc shape). The peeling portion 1123 has a spatula portion 1124 that is formed at a distal end side thereof so as to protrude in a circumferential direction, and has a gripping portion 1125 which is located closer to the proximal end side (i.e., to where the operator is located) than the spatula portion 1124 and which holds the supporting portion 1121 in an unrolled state. As is shown in FIG. 26, the peeling portion 1123 and the electrode 1120 may be introduced along the outer circumferential surface of the introduction portion 110, or may be introduced by being inserted into the main body of the introduction portion.

When the electrode 1120 is to be implanted using the peeling portion 1123, the supporting portion 1121 is unrolled and is inserted into the gripping portion 1125 from the distal end side of the gripping portion 1125. Next, as is shown in FIG. 26, the spatula portion 1124 is placed in contact with the surrounding tissue St and the peeling processing is started. Because the surrounding tissue St is generally soft, the end portion of the supporting portion 1121 that is held in the gripping portion 1125 may also be used for the peeling processing.

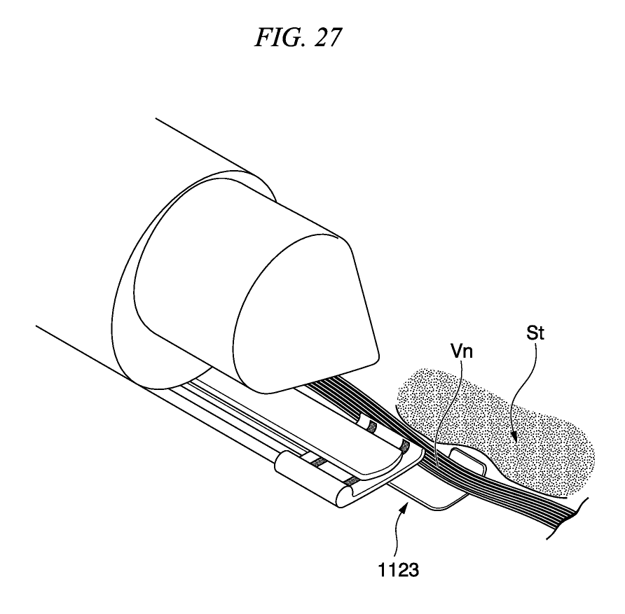

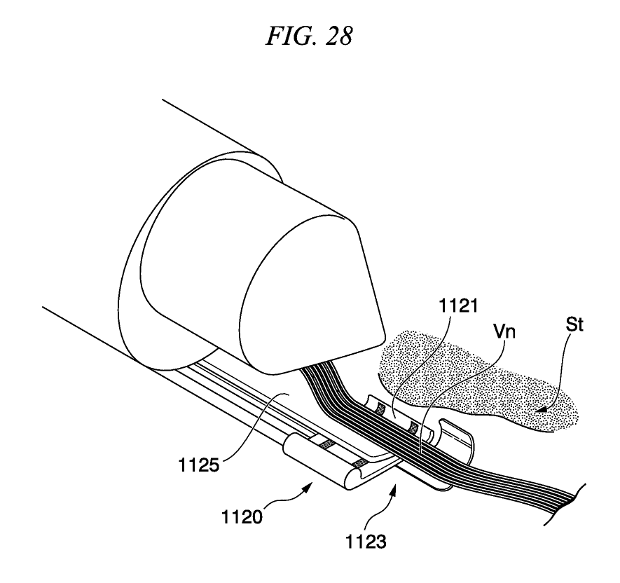

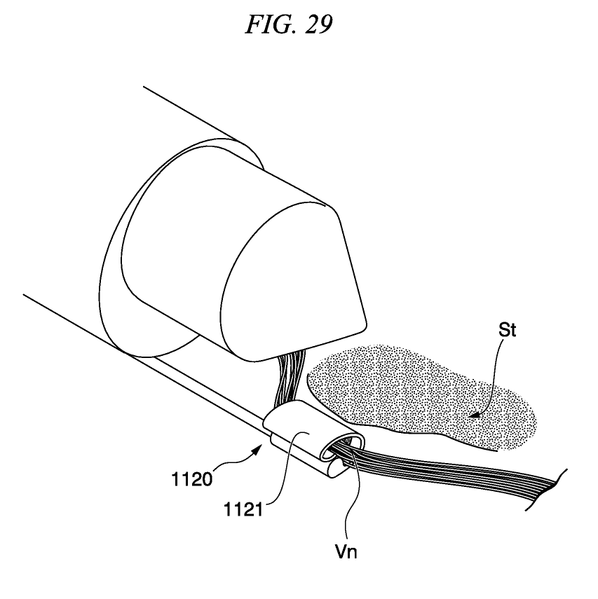

As is shown in FIG. 27, when a portion of the vagus nerve Vn is peeled away from the surrounding tissue St, the surgeon operates the peeling portion 1123 so that, as is shown in FIG. 28, the supporting portion 1121 is inserted between the surrounding tissue St and the portion of the vagus nerve Vn that is peeled away. In this state, if the engagement between the gripping portion 1125 and the supporting portion 1121 is released, the supporting portion 1121 is restored to its curved state and, as is shown in FIG. 29, the electrode 1120 is implanted in the vagus nerve Vn. Namely, in this modified example, the peeling portion 1123 doubles as an electrode manipulating component.

An appropriate method is set in order to release the engagement between the gripping portion 1125 and the supporting portion 1121. For example, it is also possible to employ a structure in which the peeling portion 1123 is formed by a first component that is provided in the spatula portion 1124, and a second component that is able to move backwards and forwards relative to the first component and that grips a supporting component between itself and the first component, and that releases the engagement between the gripping portion 1125 and the supporting portion 1121 by moving the second component backwards relative to the first component. Moreover, in the same way as in the above described embodiments, it is also possible for the stylet 140 to be inserted in the electrode 1120, and to make the gripping portion 1125 and the supporting portion 1121 move relatively in directions away from each other.

Moreover, the introduction portion in the implantation system according to the embodiments of the present invention is not limited to a configuration having a conical portion at the distal end thereof. Accordingly, the shape of the distal end portion is not particularly limited as long as it is able to bluntly dissect peripheral tissue by simply moving forward through the body interior.

Furthermore, in each of the above described embodiments, an approach is made from the superior thoracic aperture Ti so that the trachea Tc and the azygos vein Av are used as indicators, however, the embodiments of the present invention is not limited to this and peripherally located blood vessels (such as the brachiocephalic artery and the like), the esophagus, the bronchial tube, and the cardiac atrium and the like can be used as indicators.

Moreover, in each of the above described embodiments, an example is described in which the insertion portion is inserted by making a small incision in the superior thoracic aperture Ti, however, it is also possible for the introduction portion to be inserted from another location and to be introduced from that point to the vicinity of the vagus nerve Vn. An example of another location is one of the gaps between the dorsal ribs, and it is also possible to approach the vicinity of the vagus nerve Vn from here while bluntly dissecting peripheral tissue. However, because the gaps between the dorsal ribs are narrower compared with the superior thoracic aperture Ti, an introduction portion having a smaller diameter is necessary. Furthermore, because it is difficult for the trachea Tc and the azygos vein Av to be used as indicators if the approach is made via a gap between the dorsal ribs, it is preferable for the introduction portion to be moved forwards and backwards while the position thereof is confirmed using the superior vena cava SVC or radioscopic images (for example, the positions of ribs or the position of the heart as seen in radioscopic imagery) as indicators. The method used to determine the position of the distal end of the introduction portion when the superior vena cava SVC or radioscopic images are used as indicators can also be used when the superior thoracic aperture Ti is used to insert the introduction portion.

### Fourth Embodiment

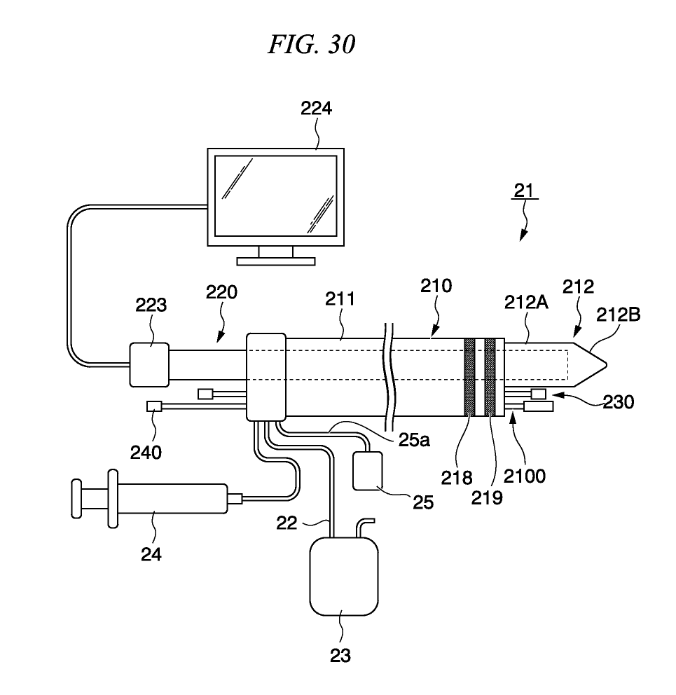

Hereinafter, a fourth embodiment of the nerve stimulation electrode implantation system of the present invention will be described with reference made to FIG. 30 through FIG. 43.

As is shown in FIG. 30, the present nerve stimulation electrode implantation system (hereinafter, this will be referred to simply as an implantation system) 21 performs various treatments in order to introduce and implant a nerve stimulation electrode (hereinafter, this will be referred to simply as an electrode) 2100 in intended nerve tissue such as a vagus nerve or the like by passing it through the inside of tissue such as connective tissue and the like. The implantation system 21 is provided with an introduction tool (i.e., introduction portion) 210 of the present embodiment that is formed in a substantially cylindrical shape, an observation portion 220 that is inserted in the introduction tool 210, a peeling portion 230 that is used to perform treatment in order for the electrode 2100 to be implanted in the subject nerve tissue, and a stylet (i.e., an electrode operating component) 240 that is used to perform the implantation operation of the nerve stimulation electrode 2100.

The introduction tool 210 is provided with a substantially circular cylinder-shaped main body 211, a transparent cap (i.e., an incision component) 212 that is attached to a distal end portion of the main body 211, and determination electrodes 218 and 219 that are provided on the external surface of the distal end portion of the main body 211.

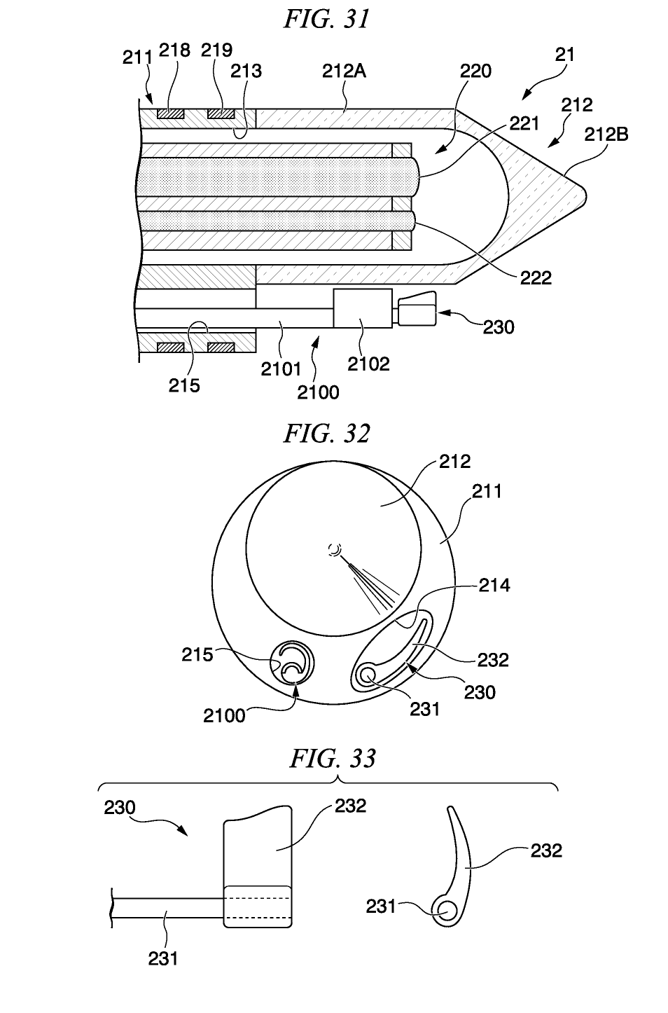

As is shown in FIG. 31 and FIG. 32, the main body 211 has three lumens (i.e., internal cavities), namely, a first lumen (i.e., a first internal cavity) 213 in which the observation portion 220 is inserted, a second lumen (i.e., a second internal cavity) 214 in which the peeling portion 230 is inserted, and a third lumen (i.e., an electrode cavity) 215 in which the electrode 2100 and the stylet 240 are inserted. A commonly known multi-lumen tube or the like can be used for the main body 211. The first lumen 213, the second lumen 214, and the third lumen 215 are formed in a substantially circular column shape.

The material used to make the main body 211 is not particularly restricted as long as it has sufficient rigidity to enable it to incise peripheral tissue (described below) and be moved forwards and backwards, and a metal such as stainless steel, or a resin or the like can be favorably used. From the standpoint of simplifying the operation to introduce the introduction tool 210, it is preferable for the outer diameter of the introduction tool 210 to be less than or equal to 6 millimeters (mm).

The cap 212 is a transparent component formed from resin or the like, and is attached to the distal end portion of the main body 211 so as to only seals off the first lumen 213. The cap 212 may be provided with an intended coloring as long as it has sufficient transparency to enable the periphery of the cap 212 to be observed from the inserted observation portion 220. The cap 212 has a circular cylinder portion 212A on the proximal end side thereof which is connected to the main body 211, and a conical portion (i.e., a blunt dissection portion) 212B which is located further to the distal end side than the circular cylinder portion 212A. The observation portion 220 which is inserted inside the first lumen 213 is able to be moved forwards and backwards through the internal cavity of the circular cylinder portion 212A, and is also able to be rotated around its own axis relative to the introduction tool 210. The surface on the distal end side of the internal cavity of the circular cylinder portion 212A is formed so as to have a predetermined curvature in order to improve the performance of the observation portion 220. The surface on the distal end side of the internal cavity of the circular cylinder portion 112A may be provided with an optical coating film may be formed thereon such that it does not obstruct the observing of illumination light (described below) of the observation portion 220.

The conical portion 212B has a radius of curvature on the distal end thereof which is set to, for example, approximately 0.2 mm, and has a blunt distal end. By directly pressing the insertion portion 210 with the conical portion 112B located in the forefront, it is possible to introduce the implantation system 21 into the vicinity of the subject tissue while bluntly incising the peripheral tissue located around the cap 112. This operation is described in further detail below in the description of the surgical procedure.

The determination electrodes 218 and 219 are formed around the entire circumference of the main body 211 from a material such as platinum that is stable inside a living organism. As is shown in FIG. 30, the determination electrodes 218 and 219 are electrically connected to a determination power supply 25 via a wire 25a that extends from the proximal end of the introduction tool 210. The determination power supply 25 is able to apply between the determination electrodes 218 and 219 a weak rectangular wave having, for example, a voltage of between 1 volt and 10 volts and a frequency of between 10 hertz and 20 hertz for between 10 microseconds and 1000 microseconds.

Note that it is also possible for the determination electrodes 218 and 219 to be connected to the nerve stimulator (described below) to which the electrode 2100 is connected, and for a voltage to be applied between the determination electrodes 218 and 219 by this nerve stimulator.

The observation portion 220 has an outer diameter that enables it to be inserted through the inside of the first lumen 213 and, as is shown in FIG. 31, the observation portion 220 has an observation optical system 221, and an illumination device 222 such as a light guide or the like that illuminates the field of view of the observation optical system 221, and these are both provided at the distal end portion of the observation portion 220. As is shown in FIG. 30, the observation portion 220 is connected to an imaging device 223 at the proximal end thereof by means of a commonly known image guide or the like. As a result, the observation portion 220 is able to obtain video images within the field of view of the observation optical system 221. The imaging device 223 is connected to a monitor 224, and video images obtained by the imaging device 223 are displayed on the monitor 224.

If the illumination device 222 is a light guide, then it is connected to a light source (not shown) which is provided within the imaging device 223. Instead of a light guide, it is also possible to mount a light-emitting component such as an LED or the like at the distal end as an illumination device.

A known endoscope device can be favorably used as the observation portion 220 as long as the dimensions of the outer diameter and the like thereof are suitable values.

FIG. 33 is an enlarged view of the distal end side of the peeling portion 230. As is shown in FIG. 32 and FIG. 33, the peeling portion 230 has an elongated rod 231, and a spatula portion 232 which is attached to a distal end portion of the rod 231. The material used to make the rod 231 is not particularly restricted provided that it has sufficient rigidity to enable it to be moved forwards and backwards in an axial direction, and a metal such as stainless steel, or a resin or the like can be favorably used.

The spatula portion 232 is formed in a plate shape or a sheet shape, and is attached such that it extends in a direction which is substantially orthogonal to the axial direction of the rod 231. The spatula portion 232 has sufficient rigidity to enable it to peel off connective tissue and the like which is peripheral to nerve tissue (hereinafter, this will be referred to as peripheral tissue), and it can be formed from the same type of material as the rod 231. A peripheral edge on the distal end side of the spatula portion 232 which extends out from the rod 231 is formed with a blunt point in the same way as the distal end of the conical portion 212B such that it is able to bluntly perform a peeling operation (described below).

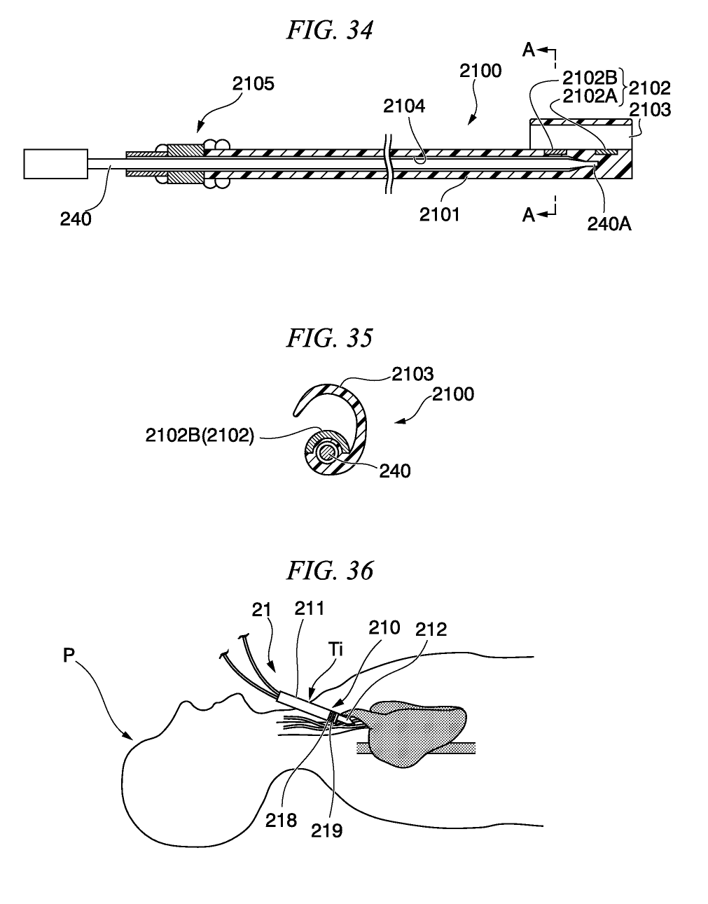

The structures of the stylet 240 and the electrode 2100 will now be described. FIG. 34 shows a partial cross-section of the electrode 2100 into which the stylet 240 is inserted. FIG. 35 is a cross-sectional view taken along a line A-A in FIG. 34.

The electrode 2100 has an elongated lead portion 2101, an electrode portion 2102 which is provided at the distal end side of the lead portion 2101, and a supporting portion 2103 which supports the electrode portion 2102 such that it makes contact with nerve tissue.

The lead portion 2101 is formed in a substantially circular cylinder shape having an internal cavity 2104 from a material such as polyurethane or the like which is highly stable inside a living organism, and the stylet 240 is able to be inserted inside the internal cavity 2104. The internal cavity 2104 is not open on the distal end side of the lead portion 2101, and the distal end portion of the internal cavity 2104 is flattened in a radial direction. As a result of this, the minimum dimension in the radial direction of this distal end portion of internal cavity 2104 is shorter than the dimension in the radial direction of the substantially circular column-shaped internal cavity 2104.

The lead portion 2101 has a plurality of leads (not shown). In each lead, conductive core wires are formed using highly durable MP35N wires or 35MLT wires. Each lead is formed with a non-conductive coating made from an ETFE material coated on each of the core wires, and these are then placed inside the internal cavity 2104. The distal end side of each lead is connected to the electrode portion 2102, while the proximal end side of each lead is connected to a connector 2105 which is used to connect to a nerve stimulator (not shown). A commonly known connector which fits the nerve stimulator to which it is connected can be suitably selected for the connector 2105. For example, an IS1 connector or the like can be used.

Note that in the above described structure, the leads may also be formed by embedding only the core wires (without non-conductive coating) inside the substantially circular cylinder-shaped peripheral wall of the lead portion 2101.

The structure of the electrode portion 2102 is not particularly restricted as long as it is able to impart electrical stimulus to nerve tissue with which it comes into contact. However, as is shown in FIG. 34, it is preferable for the electrode portion 2102 to be a bipolar type of electrode having two electrodes, namely, a negative electrode 2102A and a positive electrode 2102B. Platinum which is stable inside a living organism is used for the material for the negative electrode 2102A and the positive electrode 2102B. A titanium nitride (TiN) film having a micro-asperity structure is formed on the surfaces of the respective electrodes 2102A and 2102B so that there is reduced impedance with the surface of the living organism.

During implantation, only the side of the respective electrodes 2102A and 2102B that faces the nervous tissue is exposed. Accordingly, the surface of the respective electrodes 2102A and 2102B which is located on the opposite side is covered by silicone resin or the like, and is electrically insulated. Namely, any leakage of applied electrical energy to tissue or organs located peripherally to the nerve tissue is reduced.

If the subject nerve tissue is a vagus nerve, then it is common for the peeled-off nerve tissue to have an outer diameter of approximately 1-2 mm. Consequently, in order to fit in with the peeled-off nerve tissue, it is preferable for the core wires of the lead and the electrodes of the electrode portion to be formed to less than or equal to φ2 mm.

The supporting portion 2103 is formed by extending a portion of the silicone resin that covers a portion of the electrode portion 2102 in a substantially circular arc shape. In consideration of the function of the supporting portion 2103, it is preferable for this circular arc shape to be set such that the diameter of the circular arc is larger than the outer diameter of the nerve tissue. The supporting portion 2103 is elastically deformable, and supports nerve tissue by gripping it between itself and the electrode portion 2102. By doing this, the supporting portion 2103 is able to bring the respective electrodes 2102A and 2102B into contact with the nerve tissue. Because the shape of the supporting portion 2103 can be deformed so that it follows the outer circumferential surface of the electrode portion 2102, as is shown in FIG. 32, it is possible for the entire electrode 2100 to be housed inside the third lumen 215.

The number and configuration of the supporting portions may be suitably set in order to satisfy the aim of bringing the respective electrodes 2102A and 2102B into contact with nerve tissue.

It is also preferable for the thickness of the supporting portion to be less than or equal to 0.5 mm so that an excessive load is not applied to the subject tissue.

The stylet 240 is formed from a material such as resin or metal to dimensions that enable it to be inserted into the internal cavity 2104 of the lead portion 2101. A distal end portion 240A (see FIG. 34) of the stylet 240 is flattened in a radial direction so as to correspond to the shape of the distal end portion of the internal cavity 2104. Accordingly, if the distal end portion 240A is inserted inside the distal end portion of the internal cavity 2104 and the stylet 240 is rotated, the electrode 2100 can be rotated around the axial direction of the lead portion 2101. In addition, by moving the stylet 240 forwards and backwards in the axial direction, and by pulling the proximal end side of the lead portion 2101 back towards the operator, the electrode portion 2102 can be made to protrude from or can be retracted back into the aperture at the distal end of the third lumen 215.

A watertight component such as an O-ring or the like (not shown) is attached to the proximal end side of the second lumen 214 and the third lumen 215, and by inserting the peeling portion 230 and the electrode 2100 respectively into the second lumen 214 and the third lumen 215, the proximal end sides of the second lumen 214 and the third lumen 215 can be made watertight.

As is shown in FIG. 30, a drainage tube 22 is connected to the second lumen 214. A drainage pump 23 is connected to the drainage tube 22, and any body fluids or blood that have infiltrated the interior of the second lumen 214 from the aperture at the distal end of the second lumen 214 can be suctioned and discharged to the outside of the lumen. A syringe 24 is connected to the third lumen 215, and a physiological salt solution (i.e., saline) or the like can be supplied from the syringe 24 to the interior of the third lumen 215. As a result, saline can be released from the aperture at the distal end of the third lumen 115 so that the cap 22 can be washed, and the field of view of the observation portion 220 can be improved.

Operations when the implantation system 21 having the above described structure is put to use will now be described taking as an example a case in which the electrode 2100 is implanted in order to treat a right vagus nerve of a human (hereinafter, this will be referred to simply as a vagus nerve). When the nerve tissue adjacent to the heart which is to be stimulated is the vagus nerve, the passage from the body surface is easily distinguished from observation of the surrounding tissue. Moreover, the vagus nerve has the advantage that it is a short distance from the body surface and it is therefore easy to reach, so that access to it is comparatively easy.

A surgeon inserts the observation portion 220 into the first lumen 213 of the introduction portion 210, and the peeling portion 230 and the electrode 2100 in which the stylet 240 is inserted are inserted respectively into the second lumen 214 and the third lumen 215. A weak voltage is then maintained between the determination electrodes 218 and 219 by the determination power supply 25.

As a result, preparations before the implantation system 21 is put to use are completed. However, it is not necessarily that the peeling portion 230 and the electrode 2100 be inserted at this time. It is also possible for the peeling portion 230 and the electrode 2100 to be inserted after the introduction tool 210 is reached the vicinity of the vagus nerve. In this case, if necessary, it is preferable for stoppers to be fitted into the proximal end side of the second lumen 214 and the third lumen 215.

Next, the surgeon makes a small incision in the body surface of a patient P, and inserts the conical portion 212B of the introduction tool 210, as is shown in FIG. 30. In this example, as is shown in FIG. 36, the small incision is made adjacent to the superior thoracic aperture Ti of the patient P. Hereinafter, the example will now be described with reference made to FIG. 30.

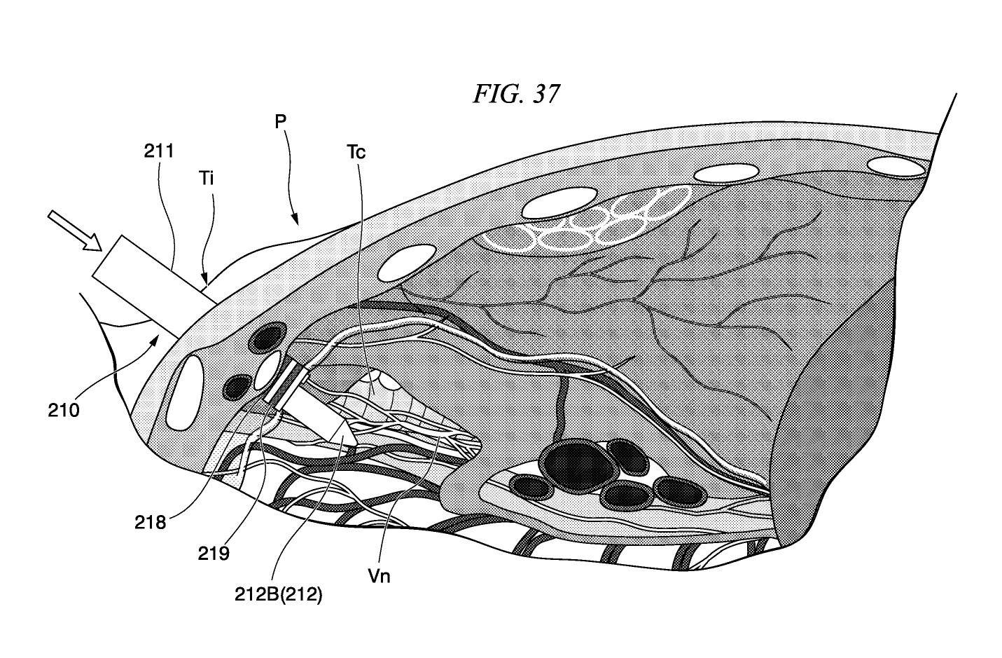

After the introduction portion 210 is inserted into the small incision, the surgeon applies force in the axial direction while holding the main body 211 and while confirming the situation surrounding the cap 212 by means of the observation portion 220, and pushes the introduction tool 210 towards the body interior. As is shown in FIG. 37, because the superior thoracic aperture Ti is located close to the trachea Tc, when the introduction tool 210 is inserted, the white tube-shaped trachea Tc shortly becomes visible within the field of view of the observation portion 220. In this example, the trachea Tc can be used as a guide to the vagus nerve Vn.

A large quantity of comparatively soft loose connective tissue is present around the introduction tool 210 in the access passage of the implantation system 21 in this example. Because of this, by pushing the introduction tool 210 forwards with the conical portion 212B in the forefront thereof, biological tissue such as the loose connective tissue and the like located in the front thereof is bluntly dissected by the conical portion 212B and the introduction tool 210 is able to move forward. Accordingly, a large amount of force is not necessary when the introduction tool 210 is being pushed in order to make it move forwards. Moreover, when the introduction tool 210 is advancing along the trachea Tc, because the boundary interface between the trachea Tc and the biological tissue around the trachea is easily split, the blunt dissection can be made to proceed even more easily. Because the periphery of the trachea Tc is covered by cartilage, as long as the distal end of the conical portion 212B is blunt, there is no possibility that the trachea Tc will be harmed by the forward movement of the introduction tool 210.

There are few blood vessels in the loose connective tissue that occupies the major portion of the biological tissue, and there is also substantially no cutting of blood vessels by the blunt conical portion 212B. Consequently, although there is no particularly large amount of bleeding while the introduction tool 210 is being moved forwards, because bodily fluids such as interstitial fluids are extravasated from the bluntly dissected biological tissue, if, necessary, these can be suctioned and expelled using the drainage pump 23.

The observation portion 220 which has been inserted into the introduction tool 210 is able to be moved forwards and backwards in an axial direction, and is also able to be rotated around its own axis. Accordingly, if a surgeon appropriately operates the observation portion 220 within the first lumen 213, that surgeon is able to obtain an excellent view of the area in front of the conical portion 212B and of the entire periphery of the cap 212 including the periphery of the circular cylinder portion 212A. Accordingly, it is possible, while confirming the biological tissue surrounding the cap 212, for a surgeon to make the distal end portion of the introduction tool 210 move forward easily as far as the vicinity of the heart where the vagus nerve Vn is located.

Before the introduction tool 210 reaches the vicinity of the vagus nerve Vn, in some cases it passes close to other nerves and the like. Although there are various types of nerve, they all appear white, linear objects, and it is difficult for a surgeon to distinguish between them.

Generally, if an electrical stimulus is applied to a nerve, a vital reaction is generated in that patient in accordance with the type of nerve. For example, it is known that if the vagus nerve Vn is stimulated, the heart rate of the patient is reduced, while if the phrenic nerve is stimulated, then convulsions are generated in the area around the abdomen of the patient.

If a surgeon causes the appropriate determination electrodes 218 and 219 to approach and come into contact with a nerve and then directly observes the vital reaction of the patient P, the surgeon is able to identify whether or not the nerve approached by the introduction tool 210 is the vagus nerve Vn.

If this nerve is the vagus nerve Vn, then the treatment described below is performed on this nerve. If, however, this nerve is not the vagus nerve Vn, then the route by which the introduction tool 210 has been moved forward is changed, and the above described procedure is repeated until the vagus nerve Vn is found.

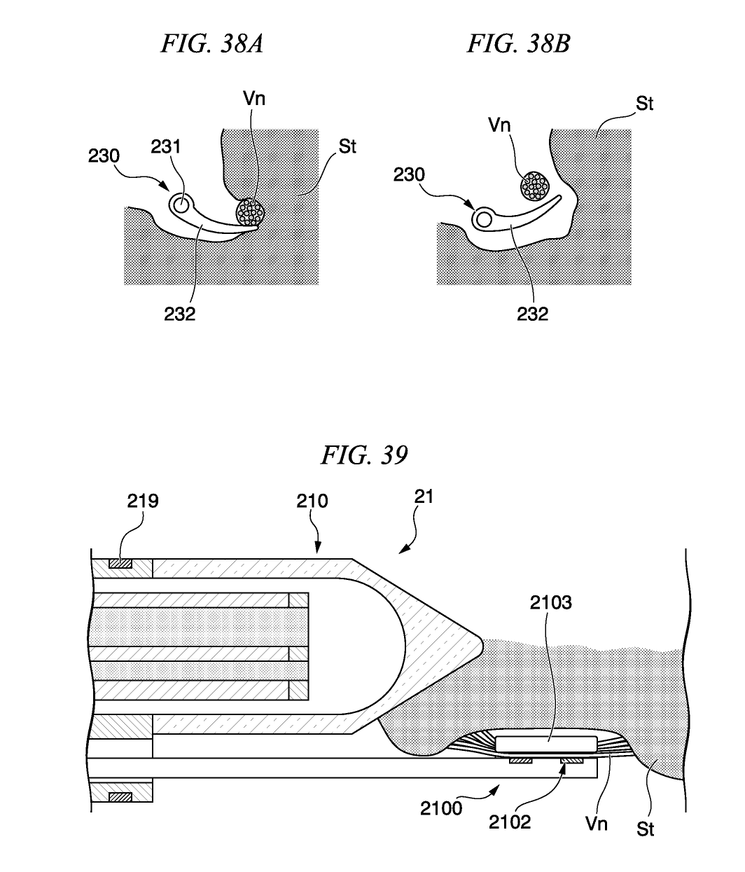

When the vagus nerve Vn is verified, the surgeon removes a portion of the surrounding tissue so as to expose a portion of the vagus nerve Vn, and also peels this portion away from the surrounding tissue such that it is possible to implant the electrode 2100. The surgeon then causes the rod 231 of the peeling portion 230 which has been inserted into the second lumen 214 to move forward and to protrude from the distal end of the introduction tool 210. If necessary, the surgeon may remove the surrounding tissue at the surface of the vagus nerve Vn using the spatula portion 232 while moving the introduction tool 210 forwards and backwards and in rotation. Because the composition of this surrounding tissue is substantially the same as that of the above-described biological tissue, it can easily be removed by the spatula 232. Thereafter, as is shown in FIG. 38A, by pushing and pulling the rod 231 while the end portion of the spatula portion 232 is pressed against the vagus nerve Vn, the surgeon is able to move the spatula portion 232 in the axial direction of the rod 231. As a result, as is shown in FIG. 38B, the vagus nerve Vn can be easily peeled away from the surrounding tissue St. After this peeling processing has ended, the surgeon re-houses the peeling portion 230 within the second lumen 214.

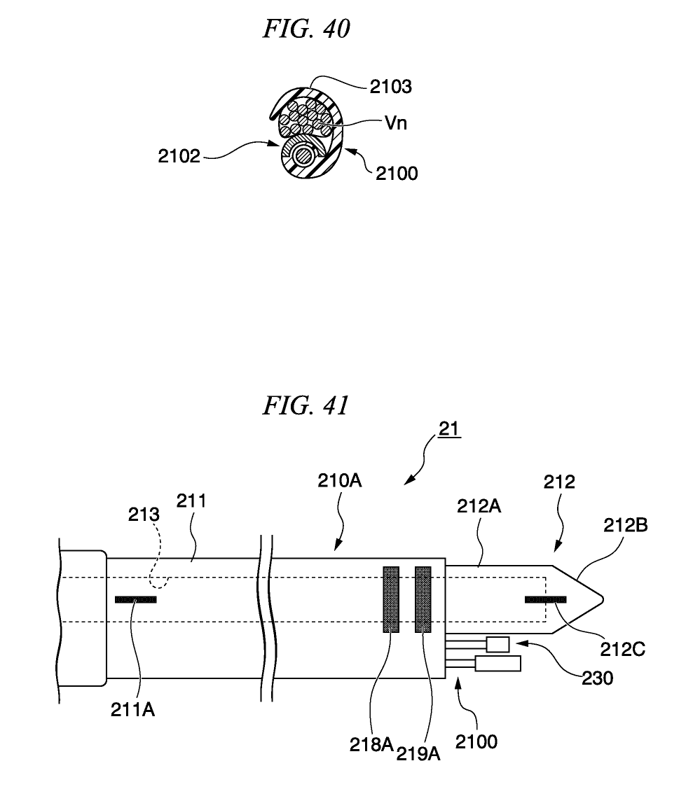

When the vagus nerve Vn peeling processing has ended, the surgeon causes the electrode 2100 inside which the stylet 240 is inserted to protrude from the third lumen 215. While adjusting the position of the supporting portion 2103 by rotating the stylet 240, the surgeon then inserts the free end of the supporting portion 2103 between the vagus nerve Vn and the surrounding tissue St such that it catches on the peeled off portion of the vagus nerve Vn. As a result, as is shown in FIG. 39 and FIG. 40, the vagus nerve Vn is supported between the supporting portion 2103 and the electrode portion 2102, and the electrode 2100 is then left implanted in the vagus nerve Vn such that the electrode portion 2102 and the vagus nerve Vn are firmly stuck together.

Note that during the peeling processing for the vagus nerve Vn and when the electrode 2100 is being implanted and so on, the field of view of the observation portion 220 can be made clearer if a physiological salt solution (i.e., saline) is injected into or suctioned from the space where this treatment is being performed using the syringe 24. It is also possible to supply carbon dioxide gas to the relevant space instead of saline, and to form a space where treatment may be performed by pushing out the surrounding tissue.

After the electrode 2100 is implanted, the surgeon extracts the stylet 240 from the electrode 2100, and withdraws the introduction tool 210 to the outside of the body by pulling it backwards. Originally, because the surrounding tissue and biological tissue completely filled the access path by which the introduction tool 210 entered the body, it accompanies the extraction of the introduction tool 210. Because of this, the path taken by the introduction tool 210 becomes completely filled up again by the surrounding tissue and biological tissue as the introduction tool 210 is withdrawn. Accordingly, after the introduction tool 210 has been withdrawn, the area surrounding the implanted electrode 2100 is also substantially filled up by the surrounding tissue and biological tissue, and the electrode 2100 becomes held in the implantation position by this surrounding tissue and biological tissue. Consequently, there is no need to provide any fixing sutures or the like after the electrode 2100 has been implanted. Moreover, because there is little movement in the vicinity of the superior thoracic aperture Ti which is caused by physical activity by the patient, the electrode position is stable.

By withdrawing the introduction tool 210, the series of tasks which are performed in order to implant the electrode 2100 is ended.

After the electrode 2100 is implanted, the connector 2105 of the electrode 2100 is connected to the nerve stimulator, and treatment by means of electrical stimulation is started. For example, a rectangular pulse voltage having a width of between several tens of microseconds (μsec) and several milliseconds (msec) is applied at a frequency of several tens of Hertz (Hz). The voltage value of the rectangular pulse voltage is appropriately set between several volts and several tens of volts. Depending on the treatment content, either continuous stimulation or intermittent stimulation may be selected, and the period for which the electrical stimulation is performed is suitably determined depending on the treatment.

After the treatment is ended, the connector 2105 is removed from the nerve stimulator, and the supporting portion 2103 is moved away from the vagus nerve Vn by pulling the end portion of the lead portion 2101. By further pulling on the center portion, the electrode 2100 can be extracted through the surrounding tissue and biological tissue to the outside of the body. Namely, because there is no need to perform any comparatively highly invasive surgical treatments in order to remove the electrode 2100, the load on the patient when the electrode is removed is considerably alleviated.

The conventional cardiac treatment apparatus described in Japanese Unexamined Patent Application, First Publication No. 2004-173790 stimulates the heart and vagus nerve Vn in accordance with the bradycardia, tachycardia, or fibrillation of the heart. Even if this cardiac treatment apparatus is used, it is not possible for the type of nerve to be identified, and a possibility that stimulation of nerves other than the treatment subject, namely, the vagus nerve Vn will increase.

According to the implantation system 21 and the introduction tool 210 of the present embodiment, a surgeon bluntly dissects biological tissue using the conical portion 212B while using the observation portion 220 to verify the area around the cap 212 that is placed at the distal end of the introduction tool 210. At this time, if necessary, the determination electrodes 218 and 219 are brought into contact with the nerve adjacent to the introduction tool 210 and a weak voltage is applied thereto. By then directly observing the vital reaction of the patient P at this time, the surgeon is able to identify whether or not the nerve to which the voltage was applied was the vagus nerve Vn. By performing peeling processing on the vagus nerve Vn using the peeling portion 230, a portion of the surrounding tissue St is removed from the vagus nerve Vn, and the electrode 2100 is implanted in the vagus nerve Vn.

By identifying the type of nerve in this manner, it is possible to limit any stimulation of nerves which are not the treatment subject, and the electrode 2100 can be implanted in the vagus nerve Vn which is the treatment subject.

If the subject tissue is the vagus nerve Vn, compared with a method in which the pleura is pierced by a trocar or the like and is accessed from inside the thoracic cavity, or a method in which a blood vessel is incised and the electrode is implanted in the blood vessel wall, not only is it possible to implant an electrode in a quicker time and with less invasiveness, but the electrode can be made to contact the vagus nerve Vn directly so that nerve stimulation treatment can be performed with a high degree of efficiency.

The determination electrodes 218 and 219 are formed around the entire circumference of the main body 211. Because of this, irrespective of which direction around the axis of the main body 211 the nerve is positioned in relative to the main body 211, it is not necessary to rotate the main body 211 around its axis, and the determination electrodes can easily be brought into contact with that nerve.

Note that the structure of the introduction tool 210 of the present embodiment can be varied in a variety of ways as is described below.

For example, as in the introduction tool 210A shown in FIG. 41, it is also possible to limit the formation of the determination electrodes 218A and 219A to only a portion in the circumferential direction of the main body 211 on the external surface of the distal end portion of the main body 211. In this case, it is possible for index marks to be formed in the same positions as the determination electrodes 218A and 218B in the circumferential direction of the first lumen 213.

Such index marks may be formed by an index mark 211A that is provided on the external surface of the proximal end portion of the main body 11, or by an index mark 212C that is provided on an external surface of the cap 212.

By forming the determination electrodes 218A and 219A of the introduction tool 210A in this manner, it is possible to easily apply voltage to only a particular nerve in the circumferential direction of the main body 211.

If the main body 211 has sufficient strength to withstand twisting, then by providing the index mark 211A on the main body 211, the surgeon is able to ascertain the position in the circumferential direction where the determination electrodes 218A and 219A are formed without directly viewing the determination electrodes 218A and 219A. In addition, by pivoting the introduction tool 210A around its own axis, it is possible to change the position in the circumferential direction where the determination electrodes 218A and 219A are formed.

In contrast, by providing the index mark 212C on the cap 212, even if the main body 211 has insufficient strength to withstand twisting, by observing the position of the index mark 212C using the observation portion 220, the positions in the circumferential direction where the determination electrodes 218A and 219A are formed can be ascertained.

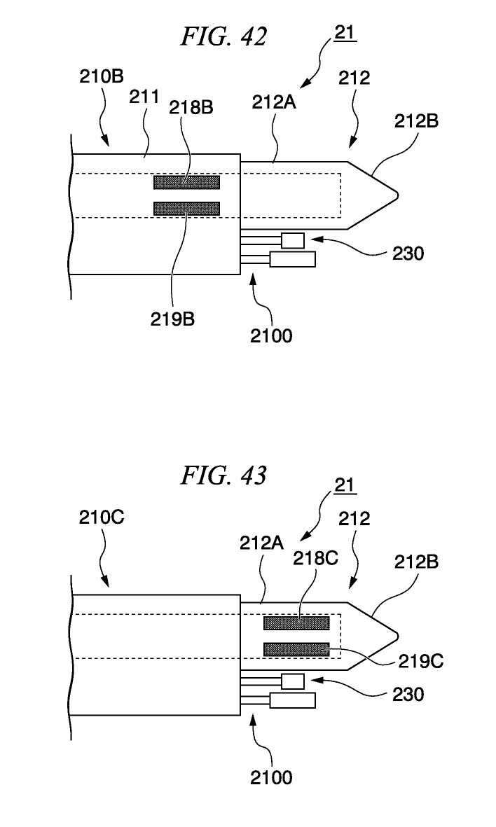

As in an introduction tool 210B shown in FIG. 42, it is also possible for determination electrodes 218B and 219B to be formed on the external surface of the distal end portion of the main body 211 such that they extend in the longitudinal direction of the main body 211.

By constructing the determination electrodes 218B and 219B of the introduction tool 210B in this manner, it is easy to bring the determination electrodes 218B and 219B into contact with a nerve, and the detection range for detecting nerves can be increased.

Moreover, as in an introduction tool 210C shown in FIG. 43, it is also possible to form determination electrodes 218C and 219C on an external surface of the cap 212.

By constructing the determination electrodes 218C and 219C of the introduction tool 210C in this manner, it is possible to observe a state in which a nerve is in contact with the determination electrodes 218C and 219C by means of the observation portion 220 which has been inserted in the first lumen 213 of the introduction tool 210C.

Note that in the implantation system 21 of the present embodiment, it is also possible for only the introduction tool 210 of this implantation system 21 to be used together with a general endoscope.

Specifically, an insertion portion of an endoscope is inserted into the first lumen 213 of the introduction tool 210, and while a surgeon is verifying the surrounding situation using the endoscope, the surgeon inserts the introduction tool 210 through a small incision that is formed in the patient P. At this time, when the insertion tool 210 approaches a particular nerve, if required, the surgeon brings the determination electrodes 218 and 219 of the introduction tool 210 into contact with that nerve and directly observes the vital reaction of the patient P, and by doing this, the surgeon is able to identify whether or not that particular nerve is the vagus nerve Vn.

In this manner, by directly observing the vital reaction of the patient P using the introduction tool 210, a surgeon is able to easily identify the type of nerve that is in contact with the determination electrodes 218 and 219, and is able to rapidly perform a surgical procedure.

### Fifth Embodiment

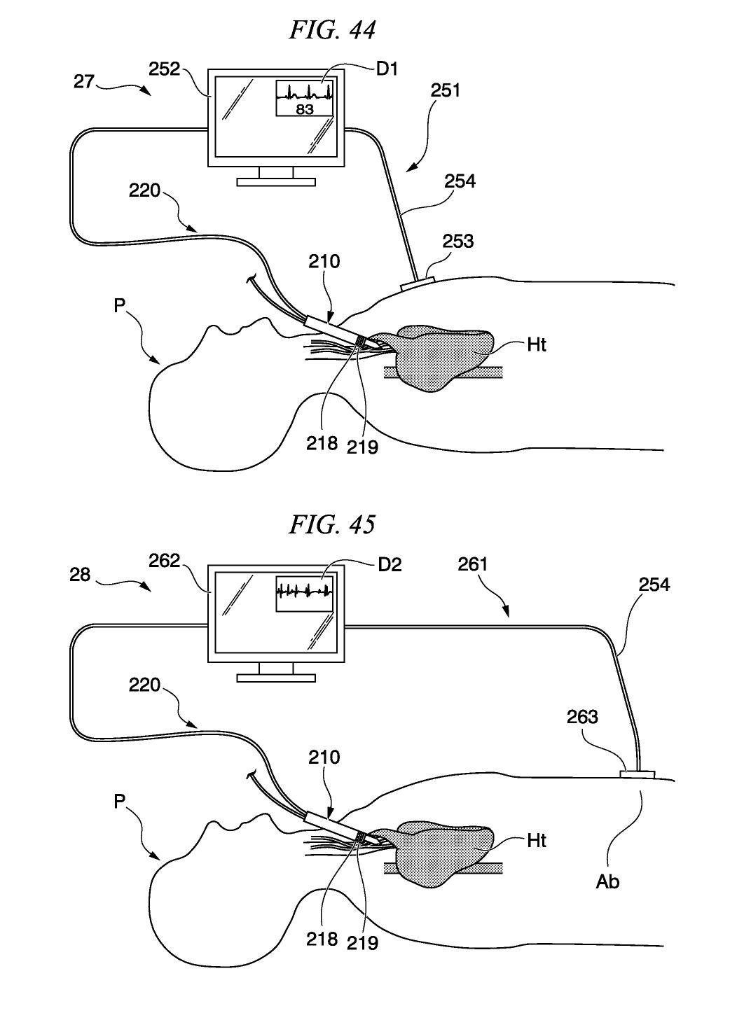

Next, a fifth embodiment of the present invention will be described with reference made to FIG. 44. Note that portions that are the same as in the above described embodiments are given the same descriptive symbols and any description thereof is omitted. Only points of difference will be described.

As is shown in FIG. 44, an implantation system 27 of the present embodiment is provided with an electrocardiogram measurement instrument (i.e., monitoring section) 251 instead of the monitor 224 of the implantation system 21 of the fourth embodiment.

The electrocardiogram measurement instrument 251 measures the electrical activity situation in the heart Ht of a patient P and displays an electrocardiogram (i.e., measurement results) D1.

Any measurement instrument having a known structure can be used for the electrocardiogram measurement instrument 251. The electrocardiogram measurement instrument 251 is provided with a display section 252 and an electrocardiogram electrode 253.

The electrocardiogram electrode 253 is attached to the surface of the body in the vicinity of the heart Ht of the patient P. Potential differences in predetermined locations of the heart Ht which are detected by the electrocardiogram electrode 253 are transmitted to a processing device (not shown) via a lead 254 and are processed, and the aforementioned electrocardiogram D1 is displayed on the display section 252.

In this example, video images obtained by the observation optical system 221 of the observation portion 220 are also able to be displayed on the display section 252.

An operation which is performed when the implantation system 27 which is constructed in this manner is put to use only differs from the operation of the above described fourth embodiment in that after the surgeon has brought the determination electrodes 218 and 219 into contact with a nerve of the patient P, the surgeon directly observes the vital reaction of the patient P and also verifies the electrocardiogram D1.

Namely, when the determination electrodes 218 and 219 of the introduction tool 210 are in contact with the vagus nerve Vn and a weak voltage is being applied to the vagus nerve Vn, intervals RR in the electrocardiogram D1 become longer, and the heart rate decreases. On the other hand, if the voltage is applied to the sympathetic nerve, the intervals RR become shorter and the heart rate increases.

In this manner, by not only observing the vital reaction of the patient P, but by also confirming the electrocardiogram D1 as well, it is possible to more reliably identify the type of nerve with which the identification electrodes 218 and 219 are in contact.

### Sixth Embodiment

Next, a sixth embodiment of the present invention will be described with reference made to FIG. 45. Note that portions that are the same as in the above described embodiments are given the same descriptive symbols and any description thereof is omitted. Only points of difference will be described.

As is shown in FIG. 45, an implantation system 28 of the present embodiment is provided with an electromyogram measurement instrument (i.e., monitoring section) 261 instead of the monitor 224 of the implantation system 21 of the fourth embodiment. The electromyogram measurement instrument 261 measures the electrical activity situation in the motor nerve of a patient P and displays an electromyogram (i.e., measurement results) D2.

Any measurement instrument having a known structure can be used for the electromyogram measurement instrument 261. The electromyogram measurement instrument 261 is provided with a display section 262 and an electromyogram electrode 263.

The electromyogram electrode 263 is attached to the abdomen Ab of the patient P. Electric potential differences in predetermined locations of the abdomen Ab which are detected by the electromyogram electrode 263 are transmitted to a processing device (not shown) via the lead 254 and are processed, and the aforementioned electromyogram D2 is displayed on the display section 262.

An operation which is performed when the implantation system 28 which is constructed in this manner is put to use only differs from the operation of the above described fourth embodiment in that after the surgeon has brought the determination electrodes 218 and 219 into contact with a nerve of the patient P, the surgeon directly observes the vital reaction of the patient P and also verifies the electromyogram D2.

Namely, when the determination electrodes 218 and 219 of the introduction tool 210 are in contact with the motor nerve and a weak voltage is being applied to the motor nerve, the muscle fibers that correspond to that motor nerve become excited so that the amplitude in the electromyogram D2 of those muscle fibers increases in size.

Before the introduction tool 210 inserted into the body of the patient P reaches the vagus nerve Vn, in some cases it crosses other nerves and the like. Nerves all appear as white, and in many cases it is difficult for a surgeon using an endoscope to distinguish between nerves and adipose tissue. In cases such as this as well, when the nerve is a motor nerve, it is possible to reliably identify the motor nerve by using the implantation system 28.

In addition, if the nerve with which the determination electrodes 218 and 219 are in contact is the motor nerve, then the route by which the introduction tool 210 has been moved forward is changed, and the above described procedure is repeated until the vagus nerve Vn is found.

The fourth embodiment through the sixth embodiment of the present invention have been described above, however, the specific structure of the present invention is not limited to the above described embodiments, and it is also possible for modifications to the structure to be made insofar as they do not depart from the spirit or scope of the present invention. Furthermore, it is to be understood that the respective component elements illustrated in the above embodiments can also be used in a variety of suitable combinations.

For example, in the above described fourth through sixth embodiments, examples have been described in which the insertion tool has three cavities, namely, a first and second lumen, and also a third lumen which is an electrode cavity, however, the number of lumens formed in the insertion tool is not limited and any suitable number may be formed.

In the above described fourth through sixth embodiments, the insertion tool has a pair of determination electrodes, however, the number of determination electrodes formed in the insertion tool can also be set to an appropriate number.

Moreover, in the above described fifth and sixth embodiments, a measurement instrument that continuously measures the blood glucose level of the patient P may also be used as the monitoring section.

### Seventh Embodiment

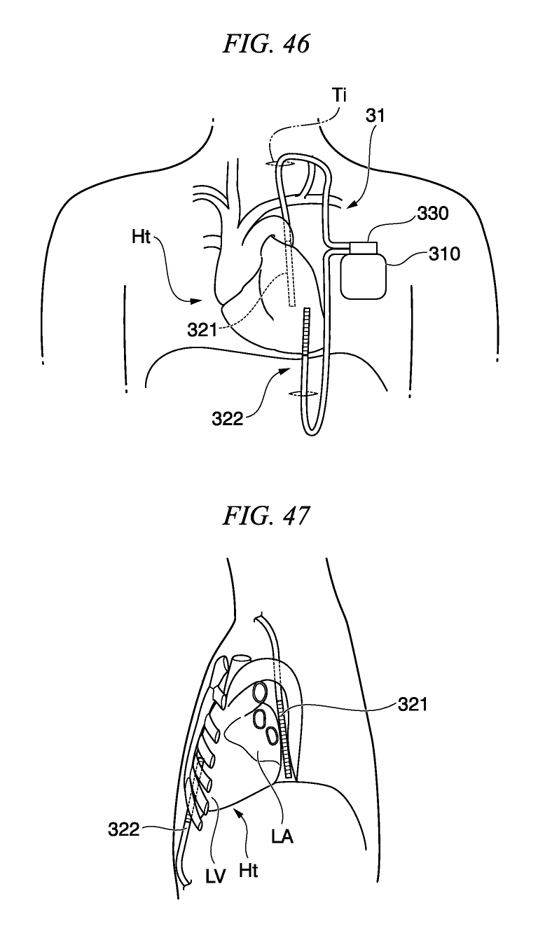

A seventh embodiment of the present invention will now be described with reference made to FIG. 46 through FIG. 58.

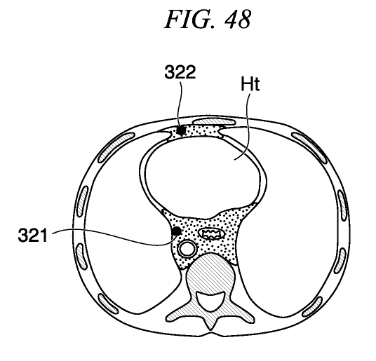

FIG. 46 through FIG. 48 show an attachment (i.e. an implantation) position where a defibrillation electrode of the present embodiment is attached. As is shown in FIG. 46 and FIG. 47, an embedded defibrillation system (hereinafter, this will be referred to simply as a defibrillation system) 31 of the present embodiment is provided with an embedded defibrillator (hereinafter, this will be referred to simply as a defibrillator) 310 that generates an electrical stimulus for defibrillation, and a pair of defibrillation electrodes 321 and 322 that are implanted peripherally to the heart Ht. The pair of defibrillation electrodes 321 and 322 correspond to the defibrillation electrode according to the embodiments of the present invention, and these are both connected to the defibrillator 310.

One of the pair of defibrillation electrodes, namely, the defibrillation electrode 321 is implanted on the rear side of the heart Ht and adjacent to the left atrium LA. The other defibrillation electrode, namely, the defibrillation electrode 322 is implanted in the precordial region of the heart Ht and adjacent to the left ventricle LV. As is shown in FIG. 48, the position where the defibrillation electrodes 321 and 322 are implanted is not in the pleural cavity, but is an interstitial space located peripherally to the heart Ht. This tissue (hereinafter, this will be referred to as the surrounding tissue) contains a large quantity of comparatively soft loose connective tissue, and in the implantation surgical procedure (described below), the implantation position can be reached comparatively easily by performing a blunt dissection.

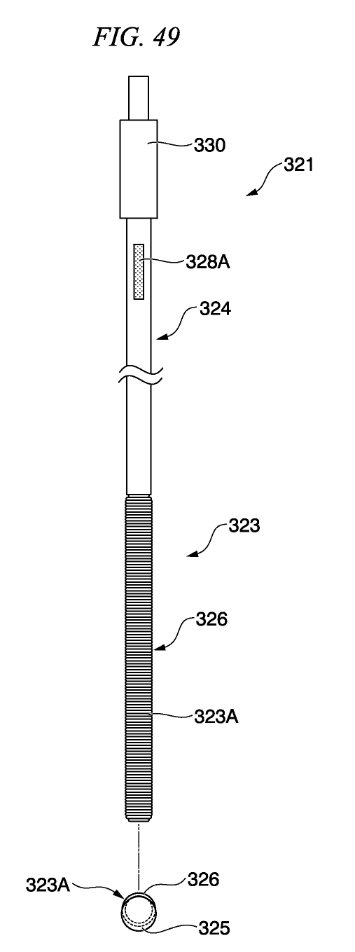

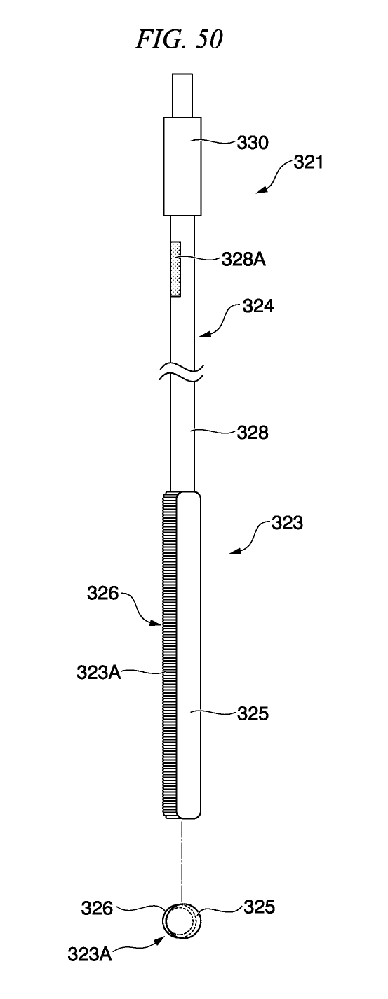

FIG. 49 is a frontal view showing the defibrillation electrode 321, while FIG. 50 is a left-side view of the same defibrillation electrode, with both of these drawings also containing a bottom view. Here, only the defibrillation electrode 321 is described, however, the structure of the defibrillation electrode 322 is the same.

The defibrillation electrode 321 is provided with an electrode portion 323 that applies electrical stimulus to a heart, and with a lead portion 324 that connects the electrode portion 323 to the defibrillator 310. The electrode portion 323 is formed by winding a highly biocompatible metallic wire, for example, a φ0.3 millimeter (mm) platinum iridium wire so as to form a coil 326, and by then covering the outer circumferential surface thereof with a covering layer 325 that is formed by a nonconductive material. A highly biocompatible material such as, for example, silicone rubber or the like can be used for the covering layer 325. The covering layer in the present embodiment is made from silicone rubber, and covers a substantially semicircular portion of the outer circumferential surface of the circular cylinder-shaped coil 326. As a result, only an approximately half-circle of the outer circumferential surface of the coil 326 is exposed so as to form an electrode surface 323A, and electrical energy is only applied from this electrode surface 323A.

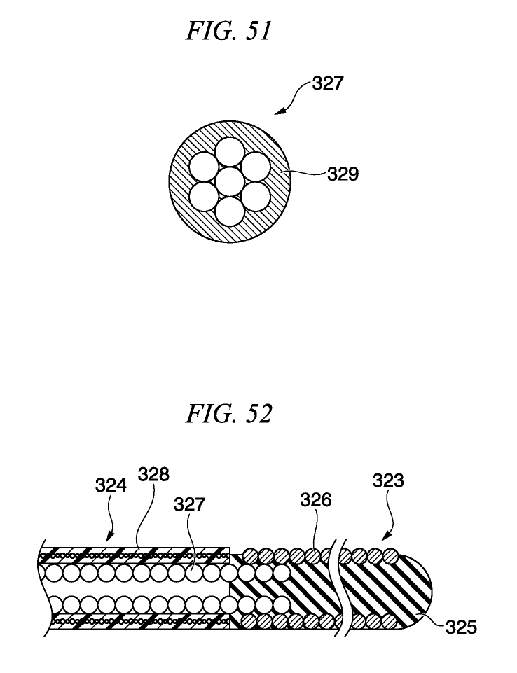

The lead portion 324 is provided with a conductive wire 327 which is wound in a coil shape, and with a non-conductive tube 328 that covers the conductive wire 327. The conductive wire 327 is made by forming commonly known 35NLT wire into 1×7 stranded wire such as is shown in FIG. 51, and the outer circumferential surface is covered by a non-conductive layer 329. Various types of resin such as, for example, ETFE and the like can be used as the material for the non-conductive layer 329. An outer diameter of the loops of the coiled conductive wire 327 is smaller than an inner diameter of the loops of the coil 326. Moreover, an end portion of the coil formed by the conductive wire 327 is pushed inside the internal cavity inside the coil 326 after the non-conductive layer 329 is removed therefrom. As a result, as is shown in FIG. 52, by then performing laser welding or the like thereon, the conductive wire 327 and the metallic wire forming the coil 326 are electrically bonded together. Note that, in FIG. 52, in order to make the drawing easier to understand, the detailed cross-sectional structure of the conductive wire 327 has not been shown. However, as is described above, the non-conductive layer 325 is removed from the portions where the conductive wire 327 and the coil 326 are connected together.

A tube made, for example, from approximately φ2 mm polyurethane or silicon rubber can be suitably used for the tube 328. As is shown in FIG. 52, because the conductive wire 327 is wound in a coil shape and is housed inside the tube 328, the lead portion 324 is highly resistant to being bent, and even if it does become bent, it is still difficult for breakages in the wire to occur. Moreover, because the rotation action when the lead portion 324 is rotated around its axis is easily transmitted to the electrode portion, it is possible to adjust the orientation of the electrode surface 323A by rotating the lead portion 324 around its axis.

An index mark 328A which is distinguishable from other portions is printed on a portion in the circumferential direction on the outer circumferential surface of the tube 328. The phase at the position where the index mark 328A is provided substantially matches the phase in the circumferential direction of the electrode surface 323A of the electrode portion 323. Accordingly, a surgeon is able to adjust the orientation of the electrode surface 323A using the index mark 328A as a guide. The conductive wire 327 extends as far as a connector 330 that is provided at a proximal end portion of the lead portion 324, and connects the defibrillator electrode 321 to the defibrillator 310 via the connector 330. Depending on the type of defibrillator and the like, connectors that are based on various standards such as IS1 and DF1 can be used where appropriate for the connector 330.

The implantation surgical procedure of the present embodiment using the defibrillation system 31 constructed in the above-described manner will now be described.

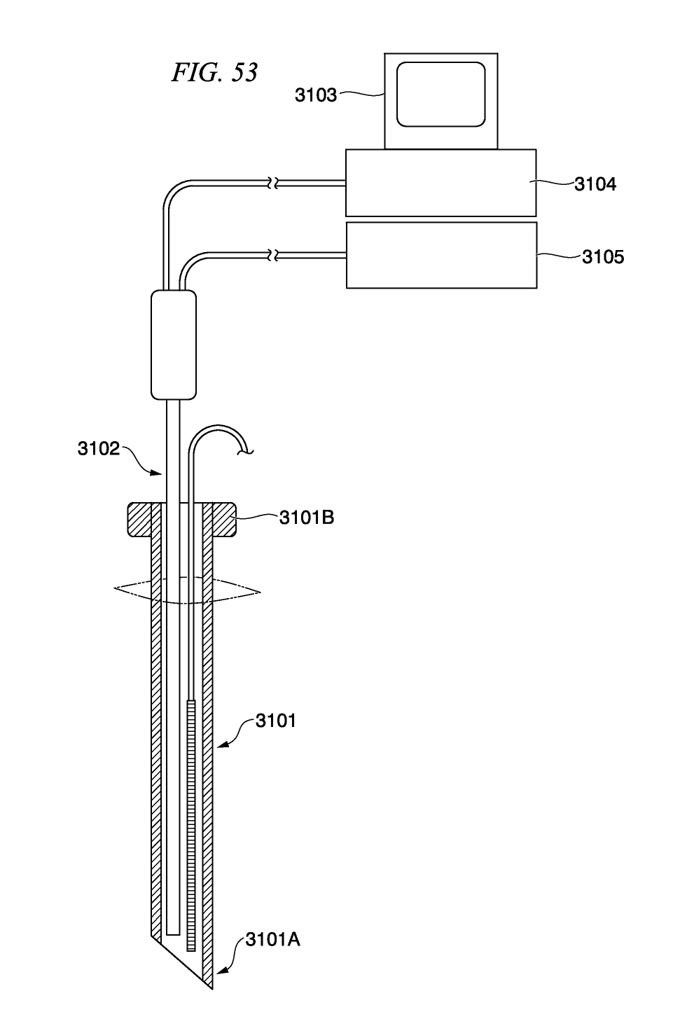

Firstly, as is shown in FIG. 53, as part of the preparatory tasks performed prior to the surgical procedure, an observation device 3102 such as an endoscope and the defibrillation electrode 321 are inserted into a cylindrical introduction component 3101. The introduction component 3101 is a circular cylinder-shaped tube that is made, for example, from resin and has an external diameter of φ8 mm, an internal diameter of φ7 mm, and a length of 200 mm. A distal end portion 3101A of the introduction component 3101, which is the first part to be introduced into a living organism, is formed as a diagonally inclined surface that slopes on an angle relative to the axis of the introduction component 3101, and a flange 3101B is formed that prevents the wrong end being inserted on a proximal end portion on the opposite side from the distal end. The material used to form the introduction component 3101 is not particularly restricted as long as it has sufficient rigidity to enable it to bluntly dissect the surrounding tissue (described below) and be moved forwards, and a resin or the like can be favorably used. Namely, in view of their dimensions and the like, commonly known trocars and the like can be appropriately used.

The observation device 3102 is a rigid endoscope having a diameter of 4 to 5 mm, and is connected to a monitor 3103, a light source 3104, and an image output amplifier 3105 and the like that are located outside the body.

The preparatory tasks are completed when the above processing is ended, however, it is not necessary for the defibrillation electrode 321 to be inserted at this time, and it is also possible for it to be inserted the introduction component 3101 has reached the vicinity of the implantation position.

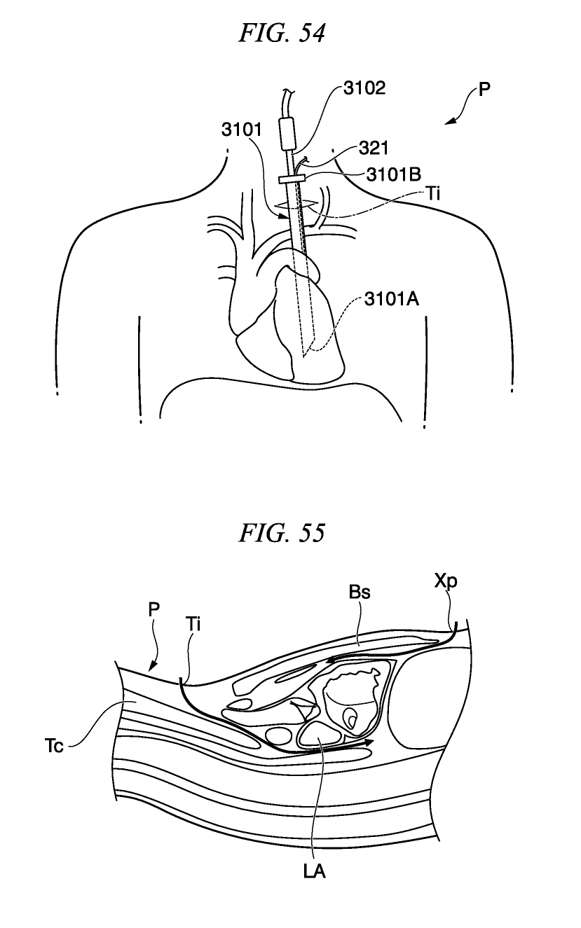

Next, as is shown in FIG. 54, the surgeon makes a small incision in the body surface adjacent to the superior thoracic aperture Ti of a patient P (i.e., in the insertion position), and inserts the distal end portion 3101A of the introduction component 3101. After the introduction component 3101 is inserted, the surgeon applies force in the axial direction while holding the flange 3101B of the introduction component 3101, and while confirming the situation in front of and surrounding the introduction component 3101 by means of the observation portion 3102, and pushes the introduction component 3101 towards the body interior.

As is shown in FIG. 55, because the superior thoracic aperture Ti is located close to the trachea Tc, when the introduction component 3101 is inserted, the white tube-shaped trachea Tc soon becomes visible within the field of view of the observation device 3102. If the introduction component 3101 is moved forward along the trachea Tc, it can easily reach the rear-side left atrium LA. Consequently, the trachea Tc can be used as a guide.

A large quantity of comparatively soft loose connective tissue is present around the introduction component 3101. Because of this, by pushing the introduction component 3101 forwards with the diagonally inclined distal end portion 3101A in the forefront thereof, the surrounding tissue located in the front thereof is bluntly dissected and the introduction component 3101 is able to move forward. Accordingly, it is not necessary to use a highly rigid member such as a metal pipe as the introduction component, and it is not necessary for a large amount of force to be used when the introduction component 3101 is being pushed in order to make it move forwards. Moreover, when the introduction component 3101 is advancing along the trachea Tc, because the boundary interface between the trachea Tc and the peripheral tissue around the trachea Tc is easily split, the blunt dissection can be made to proceed even more easily. Because the periphery of the trachea Tc is covered by cartilage, as long as the distal end portion 3101A is blunt, there is no possibility that the trachea Tc will be harmed by the forward movement thereof. Because there are few blood vessels in the loose connective tissue, and there is also substantially no cutting of blood vessels and the like provided that the distal end portion 3101A is blunt, there is no particularly large amount of bleeding while the introduction component 3101 is being moved forwards, and the level of invasiveness on the patient is also low.

The observation device 3102 which has been inserted into the introduction component 3101 is able to be moved forwards and backwards in an axial direction relative to the introduction component 3101, and is also able to be rotated around its own axis. Accordingly, if a surgeon appropriately operates the observation device 3102 within the introduction component 3101, that surgeon is able to obtain an excellent view of the entire front and periphery of the introduction component 3101. Accordingly, it is possible, while confirming the surrounding tissue and internal organs and the like, for a surgeon to make the distal end portion 3101A of the introduction component 3101 move forward easily as far as the vicinity of the rear side of the left atrium LA which is the implantation position.

When the distal end portion 3101A of the introduction component 3101 has reached the vicinity of the implantation position, the surgeon makes the defibrillation electrode 321 protrude from the aperture at the distal end of the introduction component 3101. At this time, it is also possible for the stylet (i.e., the core rod) to be inserted into the internal cavity of the lead portion 324 of the defibrillation electrode 321, thereby temporarily increasing the rigidity of the defibrillation electrode 321 and making it easier to operate.

The surgeon then rotates the defibrillation electrode 321 around its axis by an intended rotation amount while viewing the index mark 328A so that the electrode surface 323A faces towards the precordial region, and adjusts the orientation of the defibrillation electrode 321 such that it faces the left atrium LA. Note that when the defibrillation electrode 321 is being implanted and so on, the field of view of the observation device 3102 can be made clearer if a physiological salt solution (i.e., saline) is injected from the proximal end side of the introduction component 3101 using a syringe (not shown). It is also possible to supply carbon dioxide gas instead of saline. In this case, it is also possible for a watertight component such as an O-ring or the like to be fitted onto the proximal end side of the introduction component, and by placing this watertight component in tight contact with the observation device and the defibrillation electrode, to keep the proximal end side of the introduction component both airtight and watertight.

Once the orientation of the defibrillation electrode 321 has been adjusted, the surgeon holds the defibrillation electrode 321 and, at the same time, pulls the introduction component 3101 and the observation device 3102 backwards relative to the defibrillation electrode 321 so as to extract them to the outside of the body. Originally, the surrounding tissue completely filled the access path by which the introduction component 3101 advances through the body. Because of this, as the introduction component 3101 is extracted, the path taken by the introduction component 3101 becomes completely filled up again by the surrounding tissue and the like. Accordingly, after the introduction component 3101 has been extracted, the area surrounding the implanted defibrillation electrode 321 is also substantially filled up by the surrounding tissue and the like, and the defibrillation electrode 321 is implanted in the implantation position while being held there by this surrounding tissue and the like. Consequently, there is no need to provide any fixing sutures or the like in order to fix the defibrillation electrode 321 to the tissue after the defibrillation electrode 321 is implanted. Moreover, because there is little movement in the vicinity of the superior thoracic aperture Ti which is caused by physical activity by the patient, the electrode position is extremely stable.

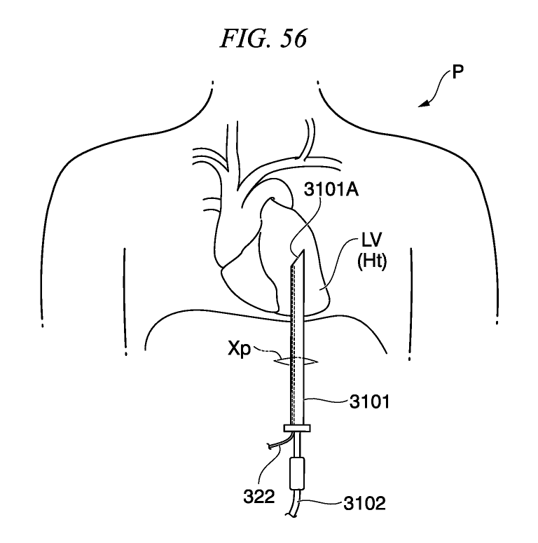

Next, the surgeon implants the other defibrillation electrode 322 using roughly the same procedure as that described above. At this time, as is shown in FIG. 56, a small incision is made in the body surface adjacent to the xiphoid process Xp of the patient P, and the distal end portion 3101A of the introduction component 3101 is inserted therein. Thereafter, the distal end portion 3101A of the introduction component 3101 is moved forward to the rear side of the ribs as it is performing blunt peeling following the line of the ribs until the distal end portion 3101A of the introduction component 3101 reaches the precordial region side adjacent to the left ventricle which is the implantation position. At this time, the surgeon is able to use the body of sternum Bs as a guide.

After the defibrillation electrodes 321 and 322 have been implanted, the connectors 330 of the lead portions of the respective defibrillation electrodes 321 and 322 are subcutaneously connected to the defibrillator 310 which is embedded under the pectoral flap. When the defibrillator 310 is subcutaneously embedded, the implantation of the defibrillation system 21 is ended.

Operations of the implanted defibrillation system 31 are roughly the same as those of a commonly known defibrillation system. Namely, the electrocardiographic waveform of the patient P is constantly monitored via the defibrillation electrodes 321 and 322, and if a predetermined waveform such as a ventricular fibrillation is detected, electrical energy is applied between the defibrillation electrodes 321 and 322 from the defibrillator 310 in order to perform defibrillation. Because the pair of defibrillation electrodes are implanted in the vicinity of the heart Ht so as to sandwich the heart Ht from the front and rear directions of the patient P, defibrillation can be performed by means of a comparatively small quantity of electrical energy.

As described above, according to the defibrillation electrodes and defibrillation system 31 of the present embodiment, because a portion of the outer circumferential surface of the coil 326 in the electrode portion 323 is covered by a non-conductive coating, only a portion in the circumferential direction of the electrode surface 323A is exposed. Because of this, by implanting the electrode surface 323A such that it faces the heart Ht, the electrical energy for performing defibrillation can be applied solely to the heart.

Accordingly, it is possible to favorably inhibit the stimulation of other tissue by the electrical energy, and prevent the electrical energy from increasing to more than the level required for the defibrillation.

In addition, the index mark 328A is provided at a position on the outer circumferential surface of the lead portion 324 that is at substantially the same phase as the electrode surface 323A. Consequently, by rotating the defibrillation electrode while viewing the index mark 328A, the surgeon is able to easily adjust the orientation of the electrode surface.

For example, if the covering layer 325 is formed from silicone rubber or the like, it is essentially not possible to view the covering layer by means of a radioscopic image. Because of this, if there is no index mark, it is extremely difficult to adjust the orientation of the electrode surface while viewing a radioscopic image. It may be possible to form the covering layer from a material created by mixing a radiopaque additive in silicone rubber, however, because the actual defibrillation electrode itself is normally small, namely, has a diameter of approximately several millimeters, it is clear that a proficiency of the surgeon, as well as a high resolution radioscopic device would be required in order to identify and adjust the orientation of the electrode surface. The defibrillation electrode according to the present embodiment of the present invention is provided with configurations which can solve this problem, and makes it possible to perform favorable implantation without proficiency or a radioscopic device having a high resolution being required.

Moreover, according to the above described defibrillation electrode implantation method, compared with the method described in Published Japanese Translation No. 2005-523786 of the PCT International Publication in which a pair of defibrillation electrodes are implanted subcutaneously so as to sandwich the heart, it is possible to implant the pair of defibrillation electrodes such that they sandwich the heart from positions which are considerably closer to the heart. Because of this, the defibrillation can be performed using a smaller amount of electrical energy, and it is possible to prolong the length of time the power supply of the defibrillation system can be left on, as well as to reduce the number of times replacements need to be provided. Furthermore, compared with a method in which the pleura is pierced by a trocar or the like and is accessed from inside the thoracic cavity, or a method in which a blood vessel is incised and the electrode is implanted in the blood vessel wall, it is possible to implant an electrode in a quicker time and with less invasiveness.

In the defibrillation electrode of the present embodiment, as the tube of the lead portion 324, it is also possible to use a tube whose rigidity has been increased by fitting a cylindrical braid that is formed from metallic wiring such as stainless steel or the like inside the wall thickness of the non-conductive tube. By employing this structure, while still retaining the insulation and flexibility of the lead portion, it is possible for a rotation operation (the torque of) performed by the surgeon to be suitably transmitted to the lead portion, and for the electrode portion to be easily rotated within the living organism.

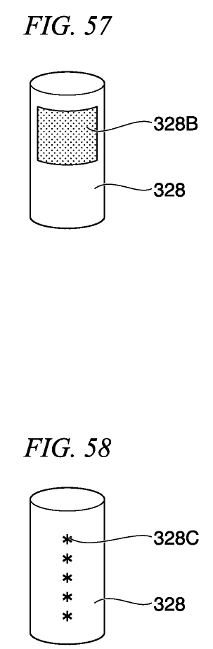

Moreover, the specific form of the index mark is not limited to that described above, and no particular restrictions are placed thereon as long as the index mark is able to be distinguished from other portions. For example, as is shown in FIG. 57, it is also possible to affix a component 328B which has a different color and to use this as the index mark. Alternatively, as is shown in FIG. 58, it is also possible to print a design 328C which has a predetermined shape and to use this as the index mark. Furthermore, it is also possible to construct an index mark that is capable of being viewed by means of radioscopy by providing an index mark on the lead portion adjacent to the electrode portion 323 using a thin plate-shaped metal component which has a predetermined shape. At this time, it is also possible for such a component to be embedded within the wall thickness of the tube so as to form an index mark that can only be viewed by means of radioscopy.

### Eighth Embodiment

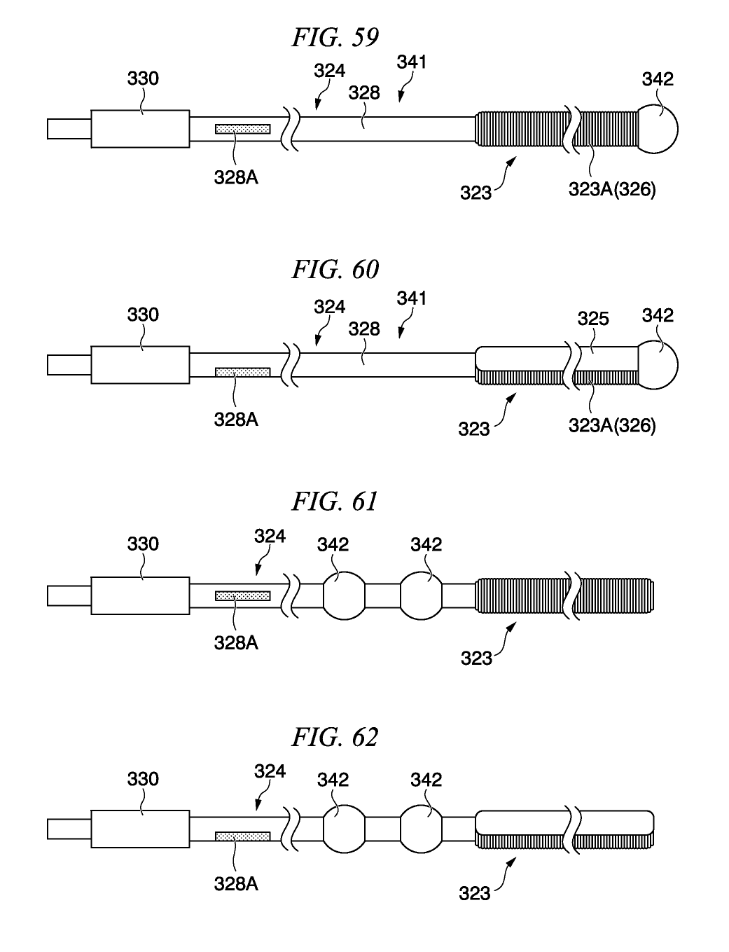

Next, an eighth embodiment of the present invention will be described with reference made to FIG. 59 through FIG. 65. The defibrillation electrode of the present embodiment differs from that described in the seventh embodiment in that a stopper is provided. Note that in the following description, structural elements that are the same as those already described are given the same symbols, and any duplicated description thereof is omitted.

FIG. 59 is a frontal view showing a defibrillation electrode 341 of the present embodiment, while FIG. 60 is a left-side view of the defibrillation electrode 341. A ball-shaped stopper 342 having a diameter of, for example, approximately 4 mm is attached to a distal end portion of an electrode portion 323, and this stopper 342 has an increased dimension in a radial direction. The stopper 342 is formed from a non-conductive material, and may be formed from the same material as that used to form a covering layer 325.

After the defibrillation electrode 341 which has the above described structure has been implanted inside a living organism via the above described surgical procedure, if any force due to physical activity or the like which may cause the defibrillation electrode 341 to withdraw from the body is applied thereto, the stopper 342 which has an enlarged dimension in the radial direction receives interference from the surrounding tissue. Because of this, the defibrillation electrode 342 is satisfactorily prevented from being extracted to the outside of the body. Accordingly, after being implanted, the electrode can be used far more stably.

The position where the stopper 342 is provided is not limited to the distal end portion of the electrode portion. For example, as is shown in the modified examples in FIG. 61 and FIG. 62, the stopper 342 may also be provided on the lead portion 324. Moreover, provided that the maximum dimension in the radial direction thereof is larger than that of the basic circular cylinder-shaped portion of the electrode portion or lead portion, then there are no particular restrictions on the shape or number of stoppers. Accordingly, the stopper may also be another shape such as a disc shape.

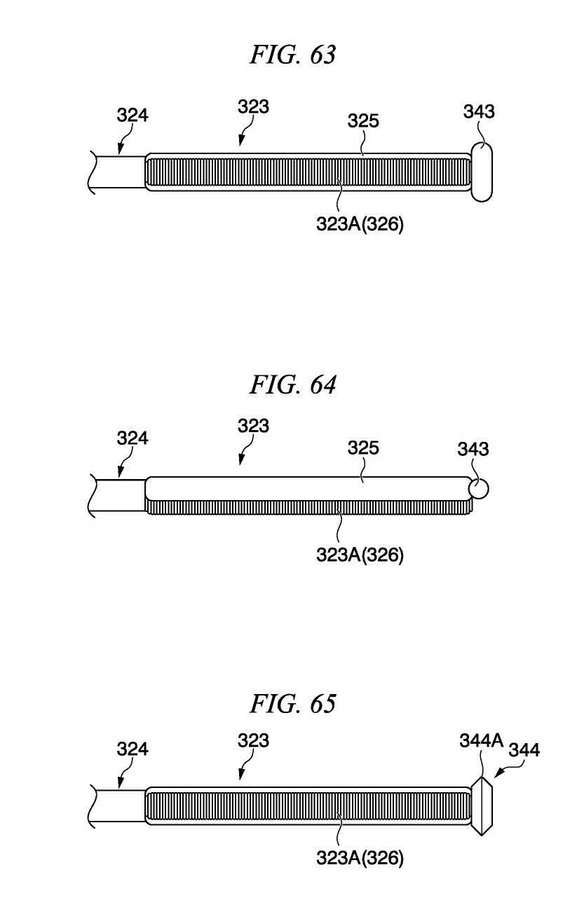

In the modified example shown in FIG. 63 and FIG. 64, a bar-shaped stopper 343 is provided on the distal end of the electrode portion 323. Because the shape of the stopper is formed to be not axially symmetrical when viewed from the axial direction of the defibrillation electrode, any movement in the axial direction of the defibrillation electrode after it has been implanted is restricted. Moreover, any rotation around its axis by the implanted defibrillation electrode can also be restricted. Accordingly, it is possible to satisfactorily suppress the orientation of the implanted electrode surface 323A changing after the implantation, and to suppress any maloccurrences such as the electrode facing away from the heart.

Moreover, the external surface of the stopper is not limited to being a curved shape, and it is also possible to provide a stopper 344 which has an edge 344A such as that shown in FIG. 65.

### Ninth Embodiment

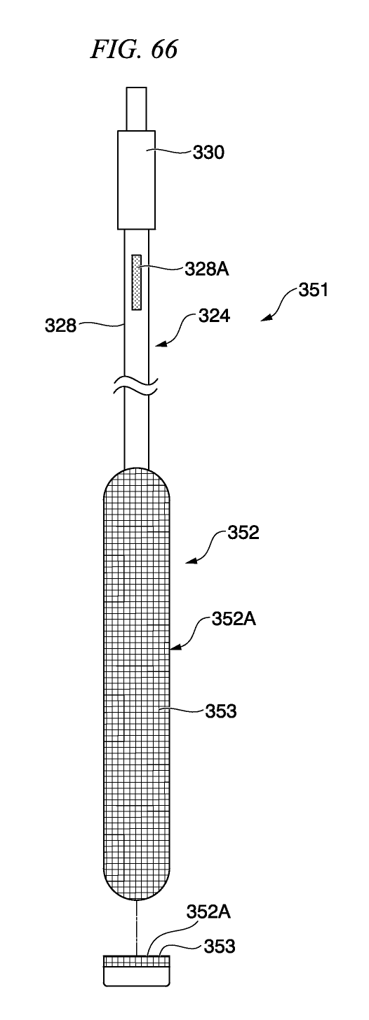

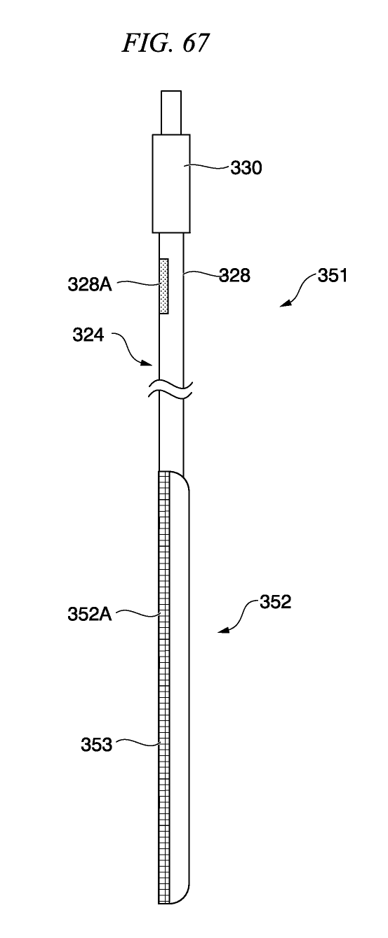

Next, a ninth embodiment of the present invention will be described with reference made to FIG. 66 and FIG. 67. The defibrillation electrode of the present embodiment differs from those of each of the above described embodiments in the shape of the electrode surface.

FIG. 66 shows a frontal view and a bottom view of a defibrillation electrode 351 of the present embodiment, while FIG. 67 shows a left-side view of the defibrillation electrode 351. An electrode portion 352 is not provided with the coil 326, and the basic-shape portion thereof is formed as a flat ellipse having, for example, a width of 4 mm and a length of 50 mm from the same material as that used to form the covering layer 325. Metallic wiring 353, which is the same as the wire forming the coil 326, is arranged in a lattice shape at a pitch of approximately 1 mm on a front surface, which is one of the surfaces in the thickness direction of the electrode portion 352, and this forms an electrode surface 352A.

When the defibrillation electrode 351 is viewed from the front, the dimension in the transverse direction of the electrode portion 352 is larger than the lead portion 324, and the surface area of the electrode surface 352A is greater than the surface area of the electrode surface 323A of the seventh embodiment. According to the defibrillation electrode 351 of the present embodiment, not only is the electrode surface greater, but by being made flat, it is possible to increase the proportion of the area of the electrode surface that faces the heart. Because of this, it is possible to apply electrical energy to the heart more efficiently.

Furthermore, because the shape of the electrode portion is not rotationally symmetrical, in the same way as in the eighth embodiment, not only is it possible to restrict any movement in the axial direction of the defibrillation electrode after it has been implanted, but also any rotation around its axis by the implanted defibrillation electrode can also be restricted.

In the present embodiment, it is not necessarily for the metallic wiring that is laid on the electrode surface to be laid in a lattice shape, and it is also possible for the metallic wiring to extend only in one predetermined direction such as, for example, a direction which is parallel to the axial direction of the defibrillation electrode, or a direction which is orthogonal to this axial direction.

Moreover, it is also possible to form the electrode surface in a concave shape having approximately the same curvature as the surface of the heart, so as to thereby further increase the proportion of the area that faces the heart.

The respective embodiments of the present invention have been described above, however, the range of technology of the present invention is not limited to the above described embodiments and it is also possible for the component elements to be used in a variety of combinations, or for various modifications to be made to the respective component elements, or for various component elements to be omitted within the spirit or scope of the present invention.

Firstly, in the defibrillation system according to the embodiments of the present invention, various modifications to the introduction component and the observation device are possible. Several modified examples of these are shown below.

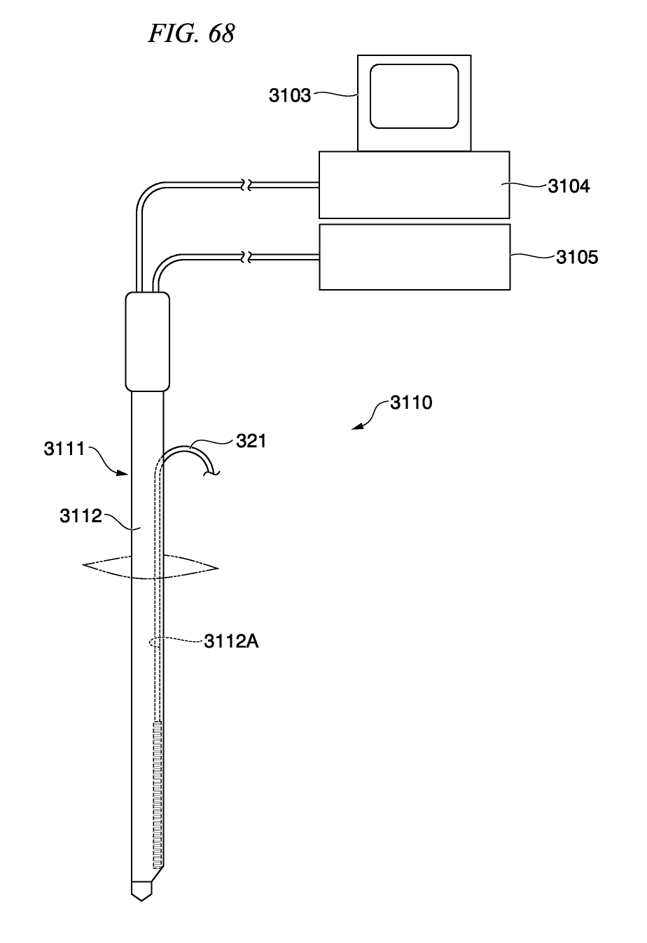

In a defibrillation system 3110 of a modified example which is shown in FIG. 68, an introduction portion 3112 of an endoscope 3111 which serves as an observation device also doubles as an introduction component. The defibrillation electrode 321 is inserted via an aperture in the proximal end side thereof into a channel 3112A that is provided in the insertion portion 3112 of the endoscope 3111, and is introduced as far as the implantation position.

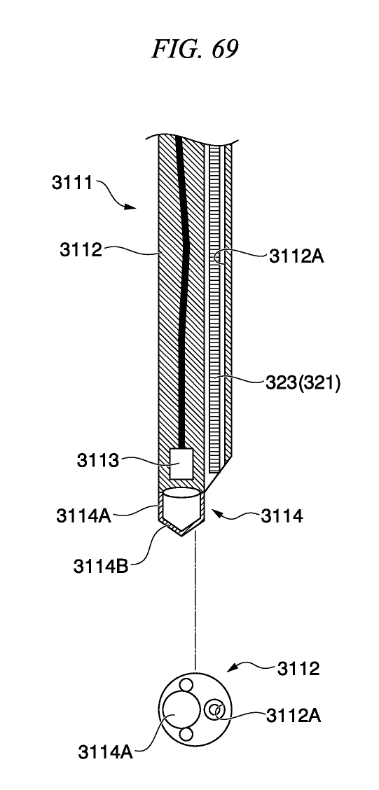

As is shown in cross-section in FIG. 69, in the endoscope 3111, a transparent cap 3114 that is formed from resin or the like is attached to a distal end of an imaging portion 3113 which is formed by a CCD or the like in order to secure a field of view within the tissue. The cap 3114 may be provided with an intended coloring as long as it still has sufficient transparency to enable the periphery of the cap 3114 to be observed from the imaging portion 3113. The cap 3114 has a circular cylinder portion 3114A on the proximal end side thereof which is connected to the insertion portion 3112, and a conical portion 3114B which is located further to the distal end side than the circular cylinder portion 3114A.

The conical portion 3114B has a radius of curvature on the distal end thereof which is set to, for example, approximately 0.2 mm, and has a blunt distal end. By directly pressing the endoscope 3111 with the conical portion 3114B located in the forefront, it is possible to introduce the endoscope 3111 into the vicinity of the implantation position while bluntly dissecting the tissue located peripherally to the cap 3114. Moreover, the distal end side of the insertion portion 3112 is cut diagonally such that the aperture of the channel 3112A is substantially parallel to the conical portion 3114B, and it is difficult for it to become caught on tissue when it is being inserted into a living organism.

In this modified example, because the insertion portion 3112 doubles as an introduction component, preparations before the operation is performed are simplified, and it is also possible to improve operability during the implantation surgical procedure.

Note that, instead of providing the imaging portion on the distal end portion of an endoscope, it is also possible to employ a structure in which observed images are transmitted via an optical fiber that has been laid in the insertion portion.

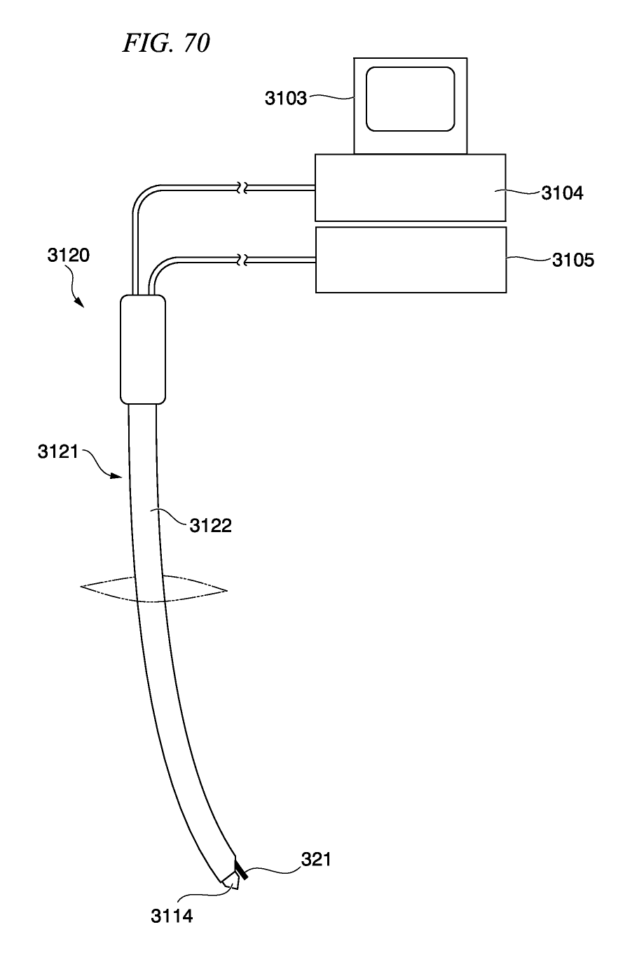

Moreover, as in a defibrillation system 3120 which is shown in FIG. 70, it is also possible for what is known as a flexible endoscope 3121 in which the insertion portion 3122 is capable of being bent to be used. If this type of endoscope is employed, then even if the path to an implantation position is bent or tortuous, the insertion portion 3122 is able to follow the path with no problems and can be smoothly introduced.

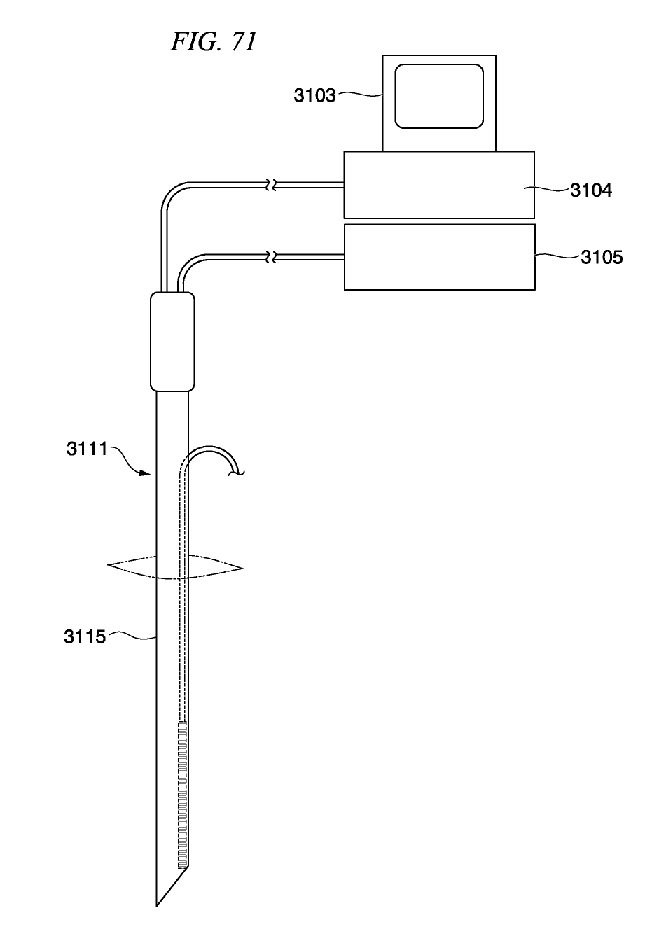

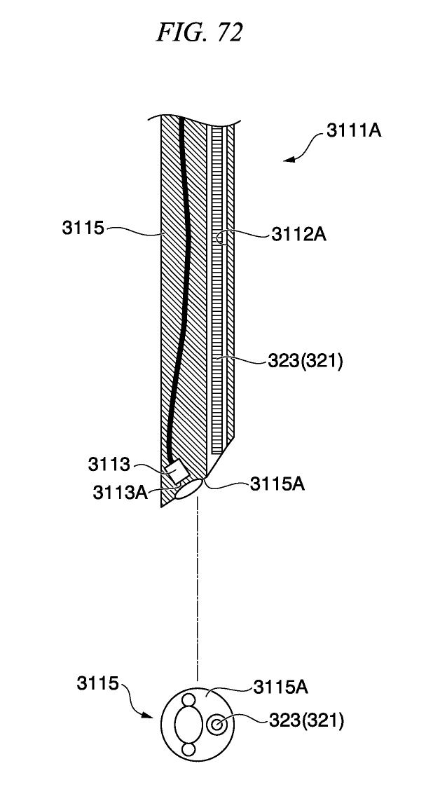

Furthermore, as is shown in FIG. 71, it is also possible for a distal end of an insertion portion 3115 of an endoscope 3111A to be formed in a diagonally inclined shape. In this case, as is shown in FIG. 72, a distal end aperture of a channel 3112A is formed in a proximal end side of an inclined surface 3115A at the distal end of the insertion portion 3115, and if the imaging portion 3113 is positioned on the distal end side of the inclined surface 3115A such that an imaging surface 3113A is parallel with the inclined surface, then the defibrillation electrode which is protruding from the distal end aperture can be easily observed, and a surgical operation can be favorably carried out.

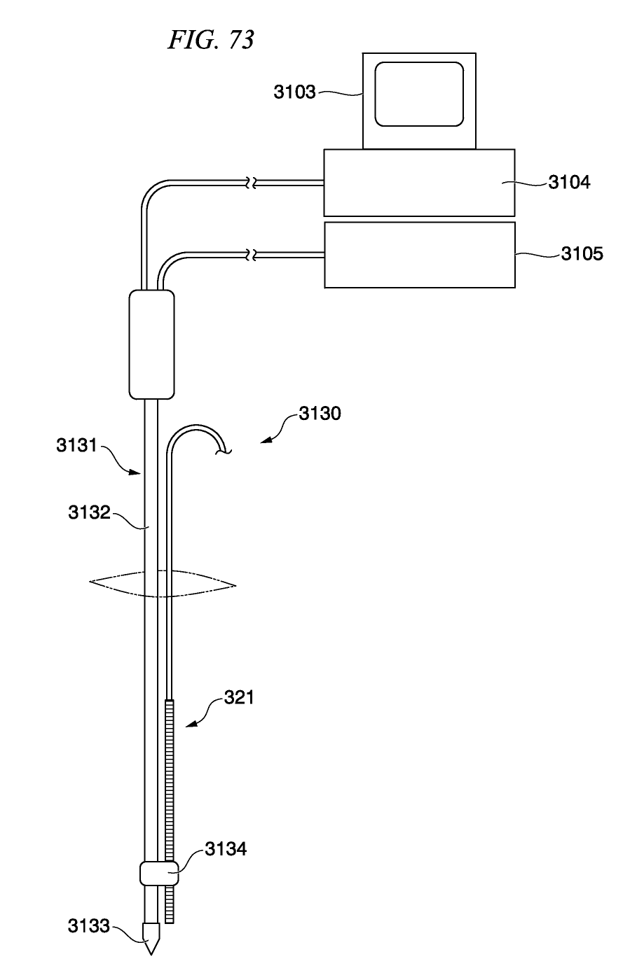

In a defibrillation system 3130 of a modified example which is shown in FIG. 73, an endoscope 3131 doubles as an introduction component. However, the defibrillation electrode 321 is not inserted into a channel, but is instead laid so as to run parallel with the insertion portion 3132 of the endoscope 3131.

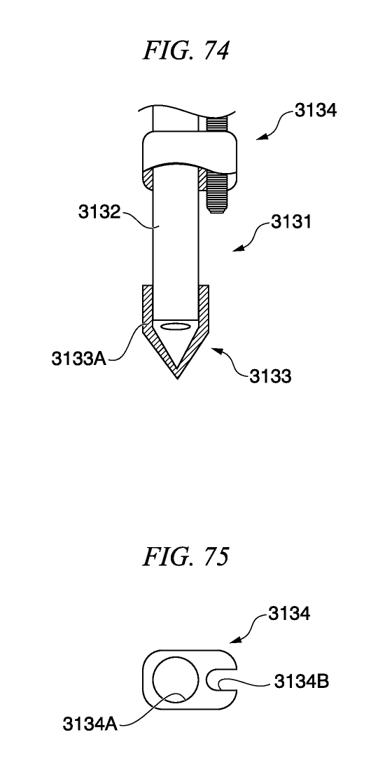

An enlargement of a distal end side of the endoscope 3131 is shown in FIG. 74. A cap 3133 of modified example that is substantially the same as the cap 3114 is fitted onto the distal end of the insertion portion 3132. Unlike the cap 3114, because the cap 3133 of the present embodiment covers the entire distal end portion of the endoscope 3132, an illumination portion (not shown) such as an LED or a light guide that illuminates the field of view of the endoscope 3132 is also positioned within the cap 3133. Accordingly, in order to protect against halation and the like of the illumination light that is irradiated from the illumination portion, if necessary, the cap 3133 may be formed such that the surface on the distal end side of the internal cavity of a circular cylinder portion 3133A has a predetermined curvature, it is also possible for an optical coating film to be formed on the cap 3133 so that the illumination light does not hinder observation.

A holder that holds a defibrillation electrode so that it runs in parallel with the insertion portion 3132 is provided in the vicinity of the distal end of the insertion portion 3132. As is shown in FIG. 75, a through hole 3134A through which the insertion portion 3132 is inserted, and a mounting groove 3134B that extends in parallel with the through hole 3134A are provided in the holder 3134. The width of the mounting groove 3134B is substantially the same as the diameter of the electrode portion 323, and by fitting the electrode portion 323 in the mounting groove 3134B, the defibrillation electrode is held such that it runs in parallel with the insertion portion.

In an implantation surgical procedure that uses this modified example, the endoscope 3131 is introduced to the vicinity of the implantation position with the electrode portion 323 already fitted into the mounting groove 3134B. When the distal end portion of the endoscope 3132 has reached the vicinity of the implantation position, the electrode portion 323 is removed from the holder 3134 by moving the defibrillation electrode 321 backward relative to the endoscope 3131. At this time, it is possible to move the endoscope 3131 forward while holding the defibrillation electrode 321. It is also possible to move the defibrillation electrode 321 backward while holding the endoscope 3131. However, moving the defibrillation electrode forwards independently may be difficult in some cases in view of its rigidity, therefore, which of these operations is to be performed may be decided after considering the positional relationship and the like between the endoscope and the implantation position.

When the engagement between the defibrillation electrode 321 and the holder 3134 is released, the surgeon rotates the insertion portion 3132 of the endoscope 3131 around its axis so as to move the holder 3134 to a position where it does not interfere with the defibrillation electrode 321, and then extracts the endoscope 3131. Thereafter, if necessary, the position and orientation of the defibrillation electrode 321 are precisely adjusted, and the implantation of the defibrillation electrode 321 is thereby completed.

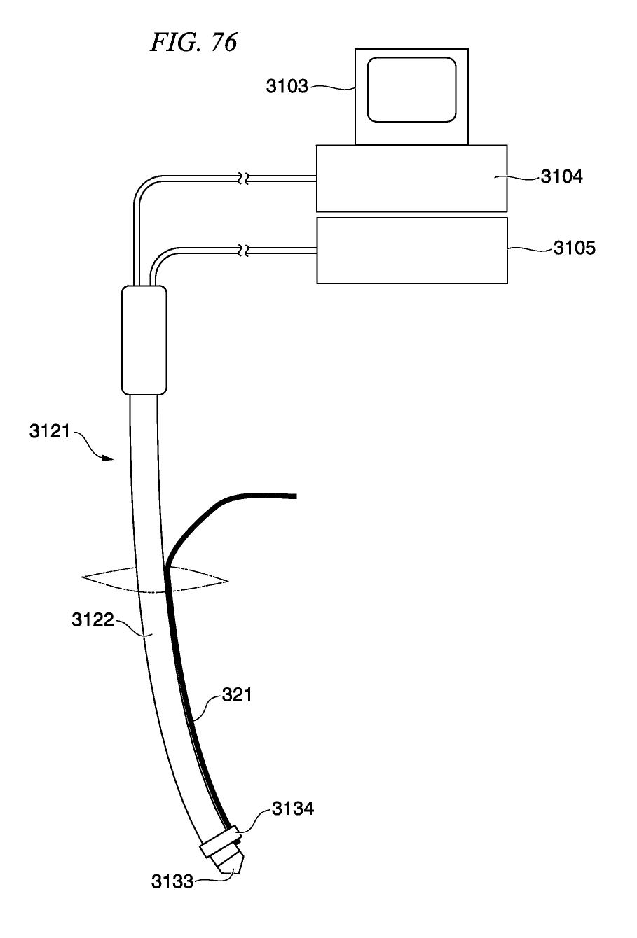

If the above described cap 3133 and holder 3134 are mounted on an existing endoscope, then as is shown in FIG. 76, it is possible to implant a defibrillation electrode while using a typical endoscope as the introduction component and observation device. As a result, there is no need to prepare a dedicated endoscope and the like, which greatly simplifies the task. Note also that, in FIG. 76, the flexible endoscope 3121 is shown as an example of an endoscope, however, the 3133 and holder 3134 may, of course, also be mounted on a rigid endoscope.

Various modifications are also possible in the electrode implantation surgical procedure as well.

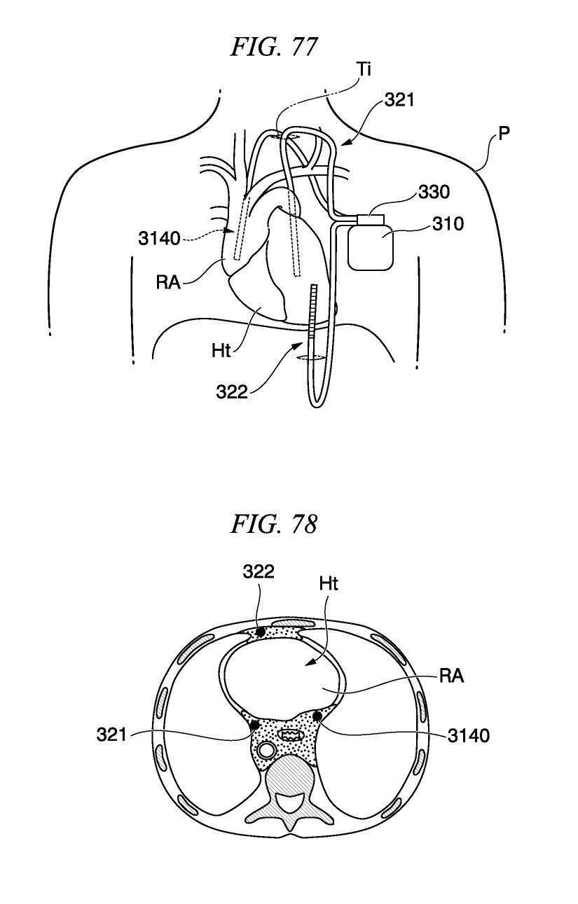

In the modified example shown in FIG. 77 and FIG. 78, two defibrillation electrodes are implanted from the superior thoracic aperture Ti side. A third defibrillation electrode 3140 that is implanted in addition to the defibrillation electrode 321 is introduced via the superior thoracic aperture Ti, and is implanted on the rear side in the vicinity of the right atrium RA. By employing this method, it is also possible to apply electrical energy to the right atrium RA side as well, and more appropriate electrical stimulus can be applied in accordance with the state of the illness.

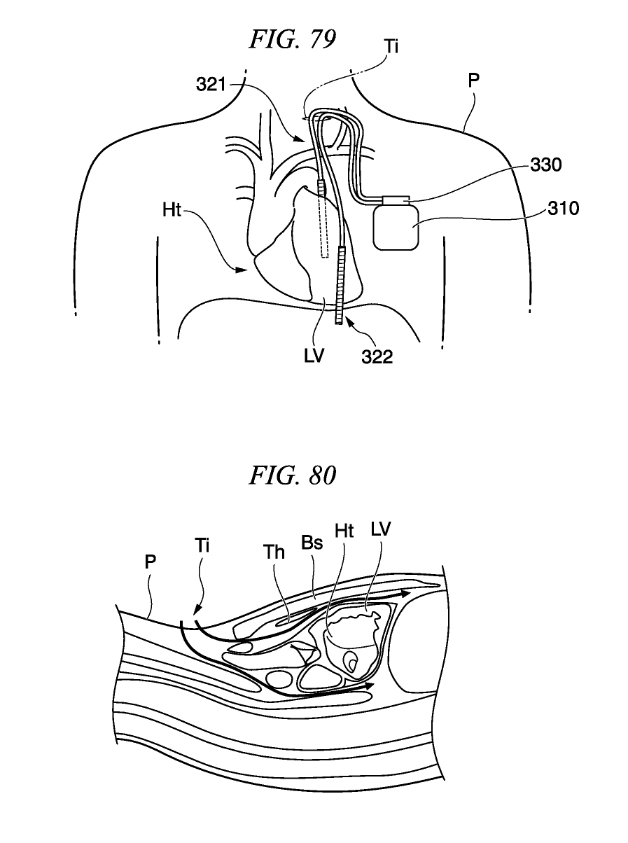

In the modified example shown in FIG. 79 and FIG. 80, both of the pair of defibrillation electrodes 321 and 322 are introduced via the superior thoracic aperture Ti. As is shown in FIG. 80, the defibrillation electrode 322 that is implanted on the precordial region side of the left ventricle LV enters on the rear side of (i.e., behind) the ribs from the superior thoracic aperture Ti, and by pushing the introduction component forward along the ribs, it can be pushed as far as the implantation position. In this case, the body of sternum Bs can be used as a guide. Note that the thymus gland Th is also present on the rear side of the body of sternum Bs. Generally, it is common for the thymus gland to be retracted in adults, however, in cases when the thymus gland is comparatively large even in an adult, and in cases when the patient is an infant, the position of the thymus gland Th is verified by means of an observation device, and care is taken such that the introduction component is introduced without injuring the thymus gland Th.

Furthermore, the present invention includes the following technological considerations.

### Additional Item 1

An approach method for approaching a target point located in a thorax using an introduction portion that has a cylindrical main body which has an internal cavity, and

an incision component which has a distal end portion that is able to bluntly dissect biological tissue, and which is attached to a distal end portion of the main body, including:

forming an insertion point for the introduction portion by incising a body surface,

inserting a distal end portion of the introduction portion at the insertion point,

forwarding the introduction portion while it bluntly dissects peripheral tissue surrounding the distal end portion,

observing the area surrounding the incision component by inserting an observation portion into the distal end portion, and

moving forward the incision portion to the vicinity of the target point.

### Additional Item 2

The approach method according to Additional item 1, further including:

identifying a type of the nerve tissue by observing the vital reaction while applying voltage to nerve tissue by a determination electrode.

### Additional Item 3

The approach method according to Additional item 1, wherein

the insertion point is formed in the superior thoracic aperture.

### Additional Item 4

The approach method according to Additional item 3, wherein

the introduction portion is moved forwards along the trachea.

### Additional Item 5

The approach method according to Additional item 2, wherein

the introduction portion is moved forwards along the body of sternum.

### Additional Item 6

The approach method according to any one of Additional items 3 to 5, further including:

determining whether or not the incision component is positioned in the vicinity of the target point by using the azygos vein as an indicator.

### Additional Item 7

The approach method according to any one of Additional items 3 to 5, further including:

determining whether or not the incision component is positioned in the vicinity of the target point by using the superior vena cava as an indicator.

### Additional Item 8

The approach method according to any one of Additional items 3 to 5, further including:

determining whether or not the incision component is positioned in the vicinity of the target point by using a radioscopic image as an indicator.

### Additional Item 9

The approach method according to Additional item 1, further including:

observing the vital reaction while verifying an electrocardiogram.

### Additional Item 10

The approach method according to Additional item 1, further including:

observing the vital reaction while verifying an electromyogram.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as limited by the foregoing description and is only limited by the scope of the appended claims.

What is claimed is:

1. A nerve stimulation electrode implantation system that is used to implant a nerve stimulation electrode in intended nerve tissue, comprising:
an introduction portion comprising:
a cylindrical main body which has an internal cavity, and
an incision component which has a blunt dissection portion that bluntly dissects biological tissue, and which is formed so as to be transparent, and which is attached to a distal end portion of the main body;
an observation portion that is inserted into the introduction portion such that it is able to observe the periphery of the incision component through the incision component; and
a peeling portion that is positioned so as to be capable of rotating around its own axis and to be capable of moving relatively in this axial direction relative to the introduction portion, the peeling portion configured to remove peripheral tissue from the periphery of the nerve tissue.

2. The nerve stimulation electrode implantation system according to claim 1, further comprising:
an electrode operating component that is inserted into the nerve stimulation electrode and configured to move the nerve stimulation electrode forwards and backwards and also in rotation, wherein
when the electrode operating component is inserted therein, the nerve stimulation electrode is introduced along the introduction portion as far as the nerve tissue.

3. The nerve stimulation electrode implantation system according to claim 1, wherein the main body comprises:
a first internal cavity inside which the observation portion is inserted so as to be capable of moving forwards and backwards; and
a second internal cavity inside which the peeling portion is inserted so as to be capable of moving forwards and backwards.

4. The nerve stimulation electrode implantation system according to claim 1, wherein the main body comprises an electrode cavity inside which the nerve stimulation electrode which has the electrode operating component inserted inside it is inserted so as to be capable of moving forwards and backwards.

5. The nerve stimulation electrode implantation system according to claim 1, wherein the field of view of the observation portion faces towards the distal end side of the introduction portion.

6. The nerve stimulation electrode implantation system according to claim 1, further comprising:
a determination electrode that is provided on at least one of an external surface of the distal end portion of the main body and an external surface of the incision portion.

7. The nerve stimulation electrode implantation system according to claim 6, wherein the determination electrode is formed around the entire circumference of the main body.

8. The nerve stimulation electrode implantation system according to claim 6, wherein the determination electrode is formed in only a portion in the circumferential direction of the main body.

9. The nerve stimulation electrode implantation system according to claim 8, wherein an index mark is formed at the same position in the circumferential direction of the internal cavity as the determination electrode on at least one of the proximal end portion of the main body and the incision component.

10. The nerve stimulation electrode implantation system according to claim 6, wherein the determination electrode is formed so as to extend in a longitudinal direction of the main body.

11. A nerve stimulation electrode implantation system that is used to implant a nerve stimulation electrode in intended nerve tissue, comprising:
an introduction portion comprising:
a cylindrical main body which has an internal cavity,
an incision component having a blunt dissection portion configured to bluntly dissect biological tissue, and which is attached to a distal end portion of the main body and is formed so as to be transparent, and
a determination electrode which is provided on at least one of an external surface of the distal end portion of the main body and an external surface of the incision portion;
an observation portion being capable of being inserted into the introduction portion so as to be capable of observing the periphery of the incision component through the incision component; and
a peeling portion that is positioned so as to be capable of rotating around its own axis and to be capable of moving relatively in this axial direction relative to the introduction portion, and that removes peripheral tissue from the periphery of the nerve tissue.

12. The nerve stimulation electrode implantation system according to claim 11, wherein the determination electrode is formed around the entire circumference of the main body.

13. The nerve stimulation electrode implantation system according to claim 11, wherein the determination electrode is formed in only a portion in the circumferential direction of the main body.

14. The nerve stimulation electrode implantation system according to claim 13, wherein an index mark is formed at the same position in the circumferential direction of the internal cavity as the determination electrode on at least one of the proximal end portion of the main body and the incision component.

15. The nerve stimulation electrode implantation system according to claim 11, wherein the determination electrode is formed so as to extend in a longitudinal direction of the main body.

16. The nerve stimulation electrode implantation system according to claim 11, further comprising:
a monitoring portion that measures the state of a living organism and displays the measurement results.

17. The nerve stimulation electrode implantation system according to claim 16, wherein the measurement results are an electrocardiogram.

18. The nerve stimulation electrode implantation system according to claim 16, wherein the measurement results are an electromyogram.

19. A defibrillation electrode comprising:
an electrode that has an electrode surface;
a lead portion whose distal end side is connected to the electrode portion, and being capable of transmitting to the electrode portion rotation movement which is applied to itself;
an index mark that is formed on a proximal end side of the lead portion on a portion in the circumferential direction of the lead portion; and
a connector that is provided on a proximal end side of the lead portion, and that is connected to an implantable defibrillator, wherein
the electrode surface is formed on a portion in the circumferential direction of the lead portion.

20. The defibrillation electrode according to claim 19, further comprising:
a stopper that is provided on at least one of the electrode portion and the lead portion, and whose maximum dimension in the radial direction of the lead portion is larger than the electrode portion and lead portion.

21. The defibrillation electrode according to claim 20, wherein the stopper has an asymmetrical shape when viewed from the axial direction of the lead portion.

22. An implantable defibrillation system comprising:
the defibrillation electrode according to claim 19;
an implantable defibrillator that is connected to the connector;
an introduction component that is able to have the defibrillation electrode either inserted inside it or mounted running alongside it, and whose distal end side is able to bluntly dissect biological tissue; and
an observation device that observes an area in front of the introduction component.

23. An approach method for approaching a target point located in a thorax using an introduction portion that has a cylindrical main body which has an internal cavity, and
an incision component which has a distal end portion that is able to bluntly dissect biological tissue, and which is attached to a distal end portion of the main body, comprising:
forming an insertion point for the introduction portion by incising a body surface,
inserting a distal end portion of the introduction portion at the insertion point,
forwarding the introduction portion while it bluntly dissects peripheral tissue surrounding the distal end portion,
observing the area surrounding the incision component by inserting an observation portion into the distal end portion, and
moving forward the incision portion to the vicinity of the target point.

24. The approach method according to claim 23, further comprising:
identifying the type of the nerve tissue by observing the vital reaction while applying voltage to nerve tissue by a determination electrode.

25. The approach method according to claim 23, wherein the insertion point is formed in the superior thoracic aperture.

26. The approach method according to claim 25, wherein the introduction portion is moved forwards along the trachea.

27. The approach method according to claim 25, wherein the introduction portion is moved forwards along the body of sternum.

28. The approach method according to claim 25, wherein whether or not the incision component is positioned in the vicinity of the target point is determined using the azygos vein as an indicator.

29. The approach method according to claim 25, wherein whether or not the incision component is positioned in the vicinity of the target point is determined using the superior vena cava as an indicator.

30. The approach method according to claim 25, wherein whether or not the incision component is positioned in the vicinity of the target point is determined using a radioscopic image as an indicator.

31. The approach method according to claim 23, wherein, in addition to the observation of the vital reaction, verification of an electrocardiogram is also performed.

32. The approach method according to claim 23, wherein, in addition to the observation of the vital reaction, verification of an electromyogram is also performed.
